(19) United States
(12) Patent Application Publication
(10) Pub. No.: US 2023/0416726 A1
(43) Pub. Date: Dec. 28, 2023

(54) Scaffolding protein functional sites using deep learning

(71) Applicant: University of Washington, Seattle, WA (US)

(72) Inventors: David BAKER, Seattle, WA (US); Jue WANG, Seattle, WA (US); Robert RAGOTTE, Seattle, WA (US); Lukas MILLES, Seattle, WA (US); Joseph L. WATSON, Seattle, WA (US); David JUERGENS, Seattle, WA (US); Sidney LISANZA, Seattle, WA (US); Amijai SARAGOVI, Seattle, WA (US); Wei YANG, Seattle, WA (US); Derrick HICKS, Seattle, WA (US); Douglas TISCHER, Seattle, WA (US); Thomas SCHLICHTHAERLE, Seattle, WA (US)

(21) Appl. No.: 18/339,519

(22) Filed: Jun. 22, 2023

Related U.S. Application Data
(60) Provisional application No. 63/366,982, filed on Jun. 24, 2022.

(51) Int. Cl. C12N 15/10; G16B 15/20; G16B 15/30; G16B 40/20

(52) U.S. Cl. CPC C12N 15/1037; C12N 15/1055; G16B 15/20; G16B 15/30; G16B 40/20

(57) ABSTRACT

De novo designed proteins with binding activity are disclosed, which were designed starting from a desired functional site and jointly filling in the missing sequence and structure needed to complete the protein, and uses of the binding proteins.

### CROSS REFERENCE

This application claims priority to U.S. Provisional Patent Application Ser. No. 63/366,982 filed Jun. 24, 2022, incorporated by reference herein in its entirety.

### FEDERAL FUNDING STATEMENT

This invention was made with government support under Grant Nos. FA8750-17-C-0219 and HR0011-21-2-0012, awarded by the Defense Advanced Research Projects Agency and Grant No. R01CA240339, awarded by the National Cancer Institute and Grant No. HHSN272201700059C, awarded by the National Institute of Allergy & Infectious Diseases and Grant No. 5U19AG065156, awarded by the National Institute on Aging. The government has certain rights in the invention.

### REFERENCE TO AN ELECTRONIC SEQUENCE LISTING

The instant application contains an electronic Sequence Listing that has been submitted electronically and is hereby incorporated by reference in its entirety. The sequence listing was created on Jun. 21, 2023, is named “22-0854-US_Sequence-Listing.xml” and is 21,575 bytes in size.

### BACKGROUND

Current approaches to de novo design of proteins harboring a desired binding or catalytic motif require pre-specification of an overall fold or secondary structure composition, and hence considerable trial and error can be required to identify protein structures capable of scaffolding an arbitrary functional site. Methods are needed to start from a desired functional site and jointly fill in the missing sequence and structure needed to complete a protein having the desired functional site.

### SUMMARY

In one aspect, the disclosure provides polypeptides comprising an amino acid sequence at least 75%, 80%, 85%, 90%, 91%, 92%, 93%, 94%, 95%, 96%, 97%, 98%, 99%, or 100% identical to the amino acid sequence selected from SEQ ID NO:1-15, wherein any N-terminal methionine residues are optional and may be present or absent. In one embodiment, the polypeptide comprises an amino acid sequence at least 75%, 80%, 85%, 90%, 91%, 92%, 93%, 94%, 95%, 96%, 97%, 98%, 99%, or 100% identical to the amino acid sequence selected from SEQ ID NO:1-8, wherein the polypeptide binds to Co²⁺. In another embodiment, the polypeptide comprises an amino acid sequence at least 75%, 80%, 85%, 90%, 91%, 92%, 93%, 94%, 95%, 96%, 97%, 98%, 99%, or 100% identical to the amino acid sequence selected from SEQ ID NO:9-10, wherein the polypeptide binds to Ca²⁺. In a further embodiment, the polypeptide comprises an amino acid sequence at least 75%, 80%, 85%, 90%, 91%, 92%, 93%, 94%, 95%, 96%, 97%, 98%, 99%, or 100% identical to the amino acid sequence selected from SEQ ID NO:11-14, wherein the polypeptide binds to Rous sarcoma virus (RSV) F protein.

In one embodiment, the polypeptide comprises an amino acid sequence at least 75%, 80%, 85%, 90%, 91%, 92%, 93%, 94%, 95%, 96%, 97%, 98%, 99%, or 100% identical to the amino acid sequence of SEQ ID NO:15, wherein the polypeptide binds to Tropomyosin receptor kinase A (TrkA). In another embodiment, the polypeptide comprises an amino acid sequence at least 75%, 80%, 85%, 90%, 91%, 92%, 93%, 94%, 95%, 96%, 97%, 98%, 99%, or 100% identical to the amino acid sequence of SEQ ID NO:22, wherein the polypeptide binds to programmed cell death ligand 1 (PD-L1).

In one embodiment, the disclosure provides nucleic acids encoding the polypeptide of any embodiment disclosed herein. In another embodiment, the disclosure provides expression vectors comprising a nucleic acid of the disclosure operatively linked to a promoter. In a further embodiment, the disclosure provides host cells comprising the nucleic acid or the expression vector of any embodiment herein.

In other aspects, the disclosure provides methods for treating cancer or generating an immune response comprising administering to a subject in need thereof a polypeptide of the disclosure. In a further aspect, the disclosure provides calcium imaging probes and electron microscopy stains, comprising a polypeptide of the disclosure.

### DESCRIPTION OF THE FIGURES

FIG. 1. Methods for protein function design. (A) Applications for functional-site scaffolding. (B-C) Design methods (LEAFEKALKEM is SEQ ID NO:16; and LEAF is SEQ ID NO:17). (C) Missing information recovery (“Inpainting”). Partial sequence and/or structural information is input into a modified RosettaFold™ network (termed RFⱼₒᵢₙₜ), and complete sequence and structure are output (LEAFEKALKEM is SEQ ID NO:16; and LEAF is SEQ ID NO:17). (D) Protein design challenges formulated as missing information recovery problems (LEAFEKALKEM is SEQ ID NO:16; and LEAF is SEQ ID NO:17). (E) Joint RosettaFold™ (RFⱼₒᵢₙₜ) can simultaneously recover structure and sequence of a masked region of protein. 2KL8 was fed into RFⱼₒᵢₙₜ with a continuous (length 30) window of sequence and structure masked out, with the network tasked with predicting the missing region of protein. Outputs closely resemble the original protein (2KL8, left) and are confidently predicted by AlphaFold™ (pLDDT/Motif RMSD of models shown: 91.6/0.91, 92.0/0.69, 90.4/0.82 respectively). (F-G) Motif scaffolding benchmarking data comparing RFⱼₒᵢₙₜ with constrained hallucination. A set of 28 de novo designed proteins, published since RosettaFold™ was trained, were used. For each protein, 20 random masks of length 30 were generated, and RFⱼₒᵢₙₜ and hallucination were tasked with filling in the missing sequence and structure to “scaffold” the unmasked “Motif”. For this mask length, RFⱼₒᵢₙₜ typically modestly outperforms hallucination, both in terms of the RMSD of the unmasked protein (the “motif”) to the original structure (F), and in AlphaFold™ confidence (pLDDT in the replaced region) (G). Circles: Average of 20 outputs for each of the benchmarking proteins. Triangle: 2KL8.

FIG. 2. Design of metal binding. (A) Di-iron binding site from E. coli cytochrome b1 (1BCF chain A residues 18-25, 27-54, 94-97, 123-130). Active site residues shown in boxes for di-iron and EF-hand respectively. (B) Absorbance spectra showing of dife_inp_1 (or mutant) in the presence (or not) of an 8-fold molar excess of Co²⁺. Note the peaks at 520 nm, 555 nm and 600 nm, consistent with Co²⁺ binding to the desired scaffolded motif (28). The mutant design was the same sequence but with the 6 coordinating residues (sidechains shown in (A)) mutated to alanine [E16A, E55A, H58A, E89A, H92A, E1 15A]). Protein concentration was 200 μM. (C) Titration analysis of Co²⁺ against the design (protein concentration=200 μM). Quantification of the absorbance at 550 nm, using a predicted extinction coefficient of 155 for Co²⁺ binding the motif (28), is consistent with both binding sites being recapitulated in the dife_inp_1 design. (D) CD spectra of design in the presence and absence of Co²⁺. Both spectra are consistent with the predicted helical structure. (E) CD melt curve in the presence and absence of Co²⁺. Note that the coordination of Co²⁺ in the protein core significantly stabilizes dife_inp_1 (protein concentration in CD experiments=6.7 μM, Co²⁺ concentration=53.3 μM). (F) AF2 prediction of inpainted design EFhand_inp_1 scaffolding the double EF-hand motif (G) Tryptophan-enhanced terbium fluorescence spectra of EFhand_inp_1 matches known spectra (59) and suggests the design can bind terbium. (H) CD spectra of EFhand_inp_1 incubated with (4×protein concentration) and without CaCl₂) suggest stabilization of the protein upon binding calcium. Design metrics (AF pLDDT, motif RMSD AF versus native): dife_inp_1 (92/0.65 Å), EFhand_inp1 (84, 0.7 Å).

FIG. 3. Design of protein-binding proteins. Designs containing target-binding interfaces built around native-complex-derived binding motifs. (A) Crystal structure of high-affinity consensus (HAC) PD-1 in complex with PD-Li. (B) Inpainted PD-L1 binder superimposed on PD-1 interface motif. (C) Max BLI binding signal versus PD-L1 concentration. (D) Crystal structure of previously designed TrkA minibinder in complex with TrkA, superimposed on TrkA receptor dimer. (E) Hallucinated bivalent TrkA binder. Protein topologies of (D-E) are shown to the right. (F) Max BLI binding signal versus TrkA concentration, showing that both binding sites bind TrkA.

FIG. 4. Training of joint sequence-structure recovery RosettaFold™. (A) Depiction of the three tasks used to train RFⱼₒᵢₙₜ, which were trained with equal likelihood (see Algorithm 1). Task 1 comprised a fixed-backbone sequence design task of a continuous segment of a given protein, without the immediate up- and downstream protein visible (see Methods; LEAFS is SEQ ID NO:18; KFEMA is SEQ ID NO:19; LFEMAGCEL is SEQ ID NO:20; and LEAFSALKFEMAGCELS is SEQ ID NO:21). Task 2 comprised an inpainting task, where the model was tasked with predicting the sequence and structure of a continuous section of protein, also without up- and downstream protein visible. Asterisks indicate “guiding points” provided as inputs during inpainting to Task 3 is the structure prediction task originally used to train RosettaFold™. (B) Training curve for RFⱼₒᵢₙₜ, showing total training and validation (crosses) losses decreasing. (C) A selection of different losses associated with each of the three tasks. RFⱼₒᵢₙₜ does not severely deteriorate in its ability to predict protein structures (task 3), but its ability to inpaint structure (task 2) improves dramatically. The model also learns to predict the sequence of a fixed backbone (task 1). (D) Masking out the structure and sequence of the flanking regions (depicted in (A), Tasks 1 and 2) improves inpainting performance. RFⱼₒᵢₙₜ was compared to an identically-trained model, except that flanking regions were not masked during training. Both AlphaFold™ pLDDT in the inpainted region (top), and the “Motif” RMSD of the AlphaFold™ predictions (bottom) were marginally better for RFⱼₒᵢₙₜ. (E) RFⱼₒᵢₙₜ outperforms RF_{implicit}, both in terms of the AlphaFold™ pLDDT in the inpainted region (left), and in the “Motif” RMSD of the AlphaFold™ prediction (right). Graphs in D and E correspond to a masked window of 30 residues.

FIG. 5. Generating diversity with inpainting. (A) With a large region of structure masked, inpainting can sometimes produce confidently-predicted designs that scaffold the input motif Two designs are shown, with the dramatically different looping order (left) or topology (right). Both designs scaffold the input “motif”. (B) Analysis performed on the inpainting benchmarking data. While the proportion of inpainted designs passing AlphaFold™ filters (>75 pLDDT, <1.5 Å) decreases with increasing size of the masked window, those designs that do pass filters, and thus successfully scaffold the motif, show more scaffold diversity (as assessed by RMSD to the native masked region) than those designs with a smaller inpainted region. (C) Further diversity can be explicitly generated by perturbing the input coordinates. During training, RFⱼₒᵢₙₜ was trained to Ca-coordinates as approximate positional information (see Methods). Therefore at inference, input Ca-coordinates can be randomly translated (uniformly sampled from within depicted spheres, left), and the model thus outputs diverse inpainted structure capable of supporting the unmasked “Motif”. All designs shown in (C) have pLDDT (both total pLDDT and just in the inpainted region)>80 and “Motif” RMSD<1.2 Å, and represent examples from each of 30 clusters (clustered at total™ score cutoff of 0.95).

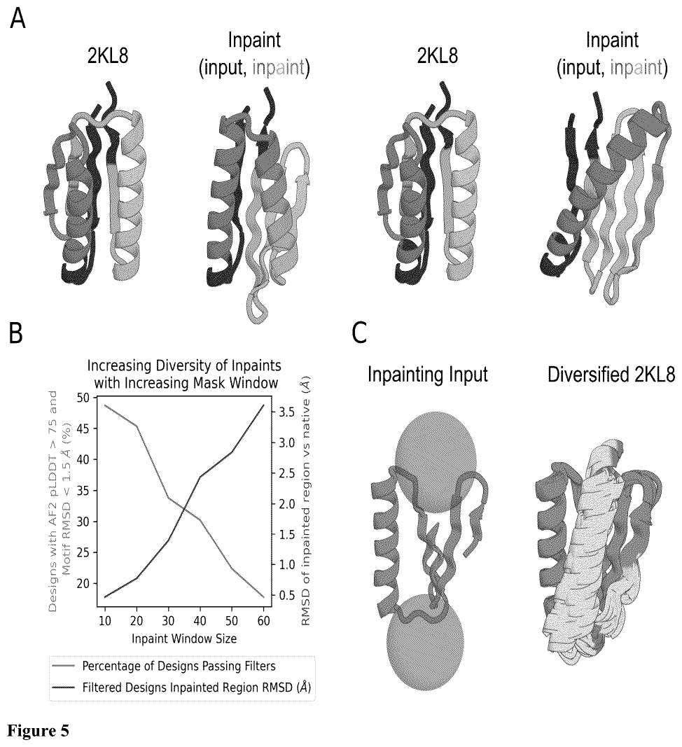

FIG. 6. A subset of successful di-iron binding proteins designed with RFⱼₒᵢₙₜ. A total of 4000 inpainted designs harboring the bacterioferritin (1BCF) di-iron binding site and encompassing 8 unique looping orders were generated with RFⱼₒᵢₙₜ. (A) 57.9% of outputs had AlphaFold™ pLDDT in the inpainted region>80 (left), and 43.7% of these designs had a predicted RMSD to the input motif<1 Å (right). (B) All 8 looping orders produced designs with AlphaFold™ pLDDT>80 and motif RMSD<1 Å. Looping orders are with respect to residue-indices in the native bacterioferritin protein (left). (C) After filtering and modest sequence optimization with RFⱼₒᵢₙₜ (see supplementary methods), 96 designs were ordered encompassing all 8 looping orders. (D-G) Characterization of three successful designs. (D) AlphaFold™ predictions of the three designs (right-most three designs), highlighting the different looping orders from the native bacterioferritin (left). Iron atoms, aligned to the motif, are depicted in gray for clarity. (pLDDT/Motif RMSD: dife_inp_1: 92/0.65 Å; dife_inp_2: 94/0.64 Å; dife_inp_3: 90/0.76 Å) (E) Designs at 200 μM were incubated with an 8×molar excess of CoCl₂. All three designs show absorbance spectra consistent with Co²⁺ binding in a tetra/penta-coordinate state to the designs. Such absorbance was not present in the absence of C2+, or with mutant designs where the 6 coordinating residues were mutated to alanine. (F) All designs showed circular-dichroism (CD) spectra consistent with helical proteins. (G) Analysis of protein stability by CD-melts. All three designs were stabilized by binding to metal ions (8× molar excess of Co²). Note that dife_inp_1 data (E-G) is the same as in FIG. 2, reproduced here for convenience.

FIG. 7. Characterization of EF-hand designs. Experimental and computational characterization of EF-hand designs tested experimentally. (A) AF2 prediction of inpainted proteins EFhand_inp_1 and EFhand_inp2 (top row) and hallucinated proteins EFhand_ha1_1 and EFhand_ha1_2 (bottom row) next to their terbium fluorescence spectra from a yeast-based initial screen (Materials and Methods). The same negative control spectrum (PDB accession 4DT5) is duplicated across all plots. (B) Computational metrics of inpainted EF-hand designs from RFⱼₒᵢₙₜ that were tested by yeast display. In addition to standard filters like motif RMSD and AF2 pLDDT, designs were also filtered by their SAP score and net charge. (C) Size exclusion chromatogram at 280 nm absorbance for EFhand_inp_1 suggests the protein occupies a stable monomeric state.

FIG. 8. Experimental characterization of inpainted PD-L1 binder. (A) Crystal structure of HAC PD-1 in complex with PD-L1 and design model of pdl1_inp_1. The overall fold of the design is quite different from HAC PD-1, as the former contains two buttressing helices against the interfacial sheet instead of the original beta-sandwich. The design also includes an additional beta strand which extends the sheet in its C-terminus. (B) The looping order of the interfacial beta strands in the design has changed dramatically from the HAC PD-1, demonstrating the ease of relooping secondary structure elements while maintaining the desired motif with inpainting. Notably, the order in which the two discontiguous strand-loop-strand submotifs appear in primary structure has switched, as well as the order in which strands 3 and 4 from HAC PD-1, which become strands 1 and 2 in the design, respectively. (C) Binding signal (PE-H) normalized to yeast surface expression (FITC-H) of clonal yeast population displaying pdl1_inp_1 labeled with 0 or 50 nM PD-L1, or 50 nM PD-L1+5 μM unlabeled PD-1. Loss of binding upon PD-1 competition suggests that pdl1_inp_1 binds PD-L1 at the native PD-1 binding site. (D) Fluorescence activated cell sorting data from yeast display binding experiments. Titles denote the concentration of a disulfide linked homodimeric PD-L1 target present in the binding reaction. Sort #1 denotes the first pooled sort of 31 designs, Sort #2 denotes the second sort performed with the enriched population of yeast displaying binding activity from Sort #1.

### DETAILED DESCRIPTION

All references cited are herein incorporated by reference in their entirety. Within this application, unless otherwise stated, the techniques utilized may be found in any of several well-known references such as: Molecular Cloning: A Laboratory Manual (Sambrook, et al., 1989, Cold Spring Harbor Laboratory Press), Gene Expression Technology (Methods in Enzymology, Vol. 185, edited by D. Goeddel, 1991. Academic Press, San Diego, CA), “Guide to Protein Purification” in Methods in Enzymology (M. P. Deutshcer, ed., (1990) Academic Press, Inc.); PCR Protocols: A Guide to Methods and Applications (Innis, et al. 1990. Academic Press, San Diego, CA), Culture of Animal Cells: A Manual of Basic Technique, 2nd Ed. (R. I. Freshney. 1987. Liss, Inc. New York, NY), Gene Transfer and Expression Protocols, pp. 109-128, ed. E. J. Murray, The Humana Press Inc., Clifton, N.J.), and the Ambion 1998 Catalog (Ambion, Austin, TX).

As used herein, the singular forms “a”, “an” and “the” include plural referents unless the context clearly dictates otherwise.

As used herein, the amino acid residues are abbreviated as follows: alanine (Ala; A), asparagine (Asn; N), aspartic acid (Asp; D), arginine (Arg; R), cysteine (Cys; C), glutamic acid (Glu; E), glutamine (Gln; Q), glycine (Gly; G), histidine (His; H), isoleucine (Ile; I), leucine (Leu; L), lysine (Lys; K), methionine (Met; M), phenylalanine (Phe; F), proline (Pro; P), serine (Ser; S), threonine (Thr; T), tryptophan (Trp; W), tyrosine (Tyr; Y), and valine (Val; V). All embodiments of any aspect of the disclosure can be used in combination, unless the context clearly dictates otherwise.

Unless the context clearly requires otherwise, throughout the description and the claims, the words ‘comprise’, ‘comprising’, and the like are to be construed in an inclusive sense as opposed to an exclusive or exhaustive sense; that is to say, in the sense of “including, but not limited to”. Words using the singular or plural number also include the plural and singular number, respectively. Additionally, the words “herein,” “above,” and “below” and words of similar import, when used in this application, shall refer to this application as a whole and not to any particular portions of the application.

In a first aspect, the disclosure provides polypeptides comprising an amino acid sequence at least 75%, 80% 85, 90%, 91%, 92%, 93%, 94%, 95%, 96%, 97%, 98%, 99%, or 100% identical to the amino acid sequence selected from SEQ TD NO:1-15 and 22, wherein any N-terminal methionine residues are optional and may be present or absent.

As described in the examples that follow, the inventors have designed a series of proteins with specific functional domains and binding activity, as described herein.

Amino acid sequences of the polypeptides are shown in Table 1.

TABLE 1

| SEQ ID | |
|---|---|
| NO. | Name/Sequence |
| 1 | >dife_inp_1 |
| | EEEEARRLLERAAELELVAINQYRRAAEEAAERAAEGDEEARELAEVERRESIDEMKHAERLR |
| | ELLARGLSPEDRPELRRALEEILRDEEGHARRYAELAEEFGSPEARELAALELDGAKLYREAL |
| | ELLS |
| 2 | >dife_inp_2 |
| | EEAKELLKELVALELDGAKRYREALEEAKAMGASEEERAVLEEILRDEEGHARRLRKYLEAGD |
| | LEGAAELARKESIDEMKHAERFAELGLPEELRELELVAINQYRKAAEALR |
| 3 | >dife_inp_3 |
| | DEELRRELERILRDEEGHAKRAREALEEAERLGLSDEVRRAAEELAALELDGAKLAREAIEEG |
| | LSEEALERYAELELVAINQYRRLAELLEREGAPEELAERFRRESIEEMKHAERLRELL |
| 4 | >dife_inp_4 |
| | EEEELEELAKELEKILRDEEGHLRKLKEALAEGLGDAEEAAELFRAESIDEMKHAEELAKLLK |
| | KGGLDPELRELLEELAELELVAINQYREAAEAAAEAAENGSEEARAAAREALEEALALELDGA |
| | KLARAALEAVEKLL |
| 5 | >dife_inp_5 |
| | LEEARAELAAILRDEEGHIRRAAAGGLTAEEFRKESIDEMKHAEKVLELAEEAAAKGDEEAAE |
| | AAAEAAELELVAINQYRAAAAAGPSEEARAALLALELDGAKLYREALAALEA |
| 6 | >dife_inp_6 |
| | DEEVEEARRALERILRDEEGHARRYEEMAEEARRMGGDEELARALEELAALELDGAKLAREAL |
| | EAVEAGDLEKAREAAEEYAELELVAINQYRELAKEYPDFAEEARRESIDEMKHAEELRELVEE |
| | L |
| 7 | >dife_inp_7 |
| | ERFEEAAELFRKESIDEMKHREELRELAEEDPEYAEIAEELAELELVAINQYGEAAEAAEEAA |
| | ENGSEEAREKALRALEEALALELDGAKRYREALEELERKGAPEEVRAALERILRDEEGHLRRL |
| | REALEELR |
| 8 | >dife_inp_8 |
| | TEELAAALEELLALELDGAKLYREALAAAPSPEERAELERILRDEEGHLAKLRELLEKFKAGA |
| | SKEELKEAAELFRRESIDEMKHAERLRELAEDPDLSPEDRAAAEEAAELELVAINQYGEAAEE |
| | AARAAE |
| 9 | >EFhand_inp_1 |
| | DEEVEKLFSLFDKDGDGTITTKELGTVFKEVAKKTGKDLPFADEEEAAKLINEVDADGNGTID |
| | FPEFLTMYKYLEENGVLDELLEALEG |
| 10 | >efhand_inp_2 |
| | MEWMTREEFKEWLEKALEDPELPISPEEAELLLELPEEELQELFEEADKDGDGTITREELEEY |
| | QKKLKELAEELLEEKK |
| 11 | >rsv_inp_1 |
| | SKKLKLYLNGKKGGSESDVNKAKSALLSTNKAVVSFSGDPELKGKEYVVELKNGKLYLKPLE |
| 12 | >rsv_inp_2 |
| | LSKEELKKALKSPEARNKAKSALLSTNKAVVSLDPDDGKTYLVSKKNGKLKVFKNGKLLVSLP |
| | L |
| 13 | >rsv_inp_3 |
| | SGGGKVNGKPISEEDRNKAKSALLSTNKAVVSVNGKEYTATKDPSTGKIVFFKNGKLVGSFPL |
| 14 | >rsv_inp_4 |
| | KKLKLTDDGLKLSEEERNKAKSALLSTNKAVVSFKSKKDGKNGLLVVSKKNGKLKVLSKLKK |
| 15 | >trkA_56 |
| | DLDIIRRAAAELKRYEEKAEEFKEHLFKQVVRLIVTPEYQANPEKLEEVIQKIKDLEEEVEKK |
| | KEELFKEVVRAIVTGDHALAERVIERARELIEEIEKKKKEISEEVELG |
| 22 | >pdl_inp_1 |
| | DLEEFFEECLKEAEEVRKEVLEGKARIQIKESVRKEDPEGGTYVCGVISLGPKGGKFHVVFHK |
| | EDPEGKEETLARFEFKVEGGKVELLELKSKDPELKKLAEKYAEKLLE |

Any N-terminal methionine residues may be present or may be absent (i.e.: deleted in the polypeptide relative to the reference polypeptide). In other embodiments, the N- and or C-terminal 1, 2, 3, 4, or 5 residues may be deleted in the polypeptide relative to the reference polypeptide.

In one embodiment, the polypeptides comprise an amino acid sequence at least 75%, 80%, 85%, 90%, 91%, 92%, 93%, 94%, 95%, 96%, 97%, 98%, 99%, or 100% identical to the amino acid sequence selected from SEQ ID NO:1-8, wherein the polypeptide binds to Co²⁺. These polypeptides may be used, for example, for binding to metals such as iron or cobalt, or other heavy metals, as part of stains for electron microscopy.

In one embodiment, the polypeptides comprise an amino acid sequence at least 75%, 80%, 85%, 90%, 91%, 92%, 93%, 94%, 95%, 96%, 97%, 98%, 99%, or 100% identical to the amino acid sequence selected from SEQ ID NO:1-3, wherein the polypeptide binds to C02+. The polypeptides are shown in the examples to have the strongest Co²⁺-binding activity. In one embodiment, the residues highlighted in boldface font in Table 1 are conserved (i.e., identical) in the polypeptides. These residues are particularly useful for binding to Co²⁺.

In another embodiment, the polypeptides comprise an amino acid sequence at least 75%, 80%, 85%, 90%, 91%, 92%, 93%, 94%, 95%, 96%, 97%, 98%, 99%, or 100% identical to the amino acid sequence selected from SEQ ID NO:9-10, wherein the polypeptide binds to Ca²⁺.

These polypeptides may be used, for example, in calcium imaging probes.

In a further embodiment, the polypeptides comprise an amino acid sequence at least 75%, 80%, 85%, 90%, 91%, 92%, 93%, 94%, 95%, 96%, 97%, 98%, 99%, or 100% identical to the amino acid sequence selected from SEQ ID NO:11-14, wherein the polypeptide binds to Rous sarcoma virus (RSV) F protein. These polypeptides may be used, for example, as epitope-based vaccines. In some embodiments, the polypeptides of this embodiment bind to preRSVF and RSVF-site V immunogens.

In one embodiment, the polypeptides comprise comprising an amino acid sequence at least 75%, 80%, 85%, 90%, 91%, 92%, 93%, 94%, 95%, 96%, 97%, 98%, 99%, or 100% identical to the amino acid sequence of SEQ ID NO:15, wherein the polypeptide binds to Tropomyosin receptor kinase A (TrkA). These polypeptides may be used, for example, as cancer therapeutics.

In one embodiment, the polypeptides comprise an amino acid sequence at least 75%, 80%, 85%, 90%, 91%, 92%, 93%, 94%, 95%, 96%, 97%, 98%, 99%, or 100% identical to the amino acid sequence of SEQ ID NO:22, wherein the polypeptide binds to programmed cell death ligand 1 (PD-L1). These polypeptides may be used, for example, as cancer therapeutics.

In one embodiment, substitutions relative to the reference sequence are conservative amino acid substitutions. As used herein, a “conservative amino acid substitution” means a given amino acid can be replaced by a residue having similar physiochemical characteristics, e.g., substituting one aliphatic residue for another (such as Ile, Val, Leu, or Ala for one another), or substitution of one polar residue for another (such as between Lys and Arg; Glu and Asp; or Gln and Asn). Other such conservative substitutions, e.g., substitutions of entire regions having similar hydrophobicity characteristics, are known. Amino acids can be grouped according to similarities in the properties of their side chains (in A. L. Lehninger, in Biochemistry, second ed., pp. 73-75, Worth Publishers, New York (1975)): (1) non-polar: Ala (A), Val (V), Leu (L), Ile (I), Pro (P), Phe (F), Trp (W), Met (M); (2) uncharged polar: Gly (G), Ser (S), Thr (T), Cys (C), Tyr (Y), Asn (N), Gln (Q); (3) acidic: Asp (D), Glu (E); (4) basic: Lys (K), Arg (R), His (H). Alternatively, naturally occurring residues can be divided into groups based on common side-chain properties: (1) hydrophobic: Norleucine, Met, Ala, Val, Leu, Ile; (2) neutral hydrophilic: Cys, Ser, Thr, Asn, Gln; (3) acidic: Asp, Glu; (4) basic: His, Lys, Arg; (5) residues that influence chain orientation: Gly, Pro; (6) aromatic: Trp, Tyr, Phe. Particular conservative substitutions include, but are not limited to, Ala into Gly or into Ser; Arg into Lys; Asn into Gln or into H is; Asp into Glu; Cys into Ser; Gln into Asn; Glu into Asp; Gly into Ala or into Pro; His into Asn or into Gln; Ile into Leu or into Val; Leu into Ile or into Val; Lys into Arg, into Gln or into Glu; Met into Leu, into Tyr or into Ile; Phe into Met, into Leu or into Tyr; Ser into Thr; Thr into Ser; Trp into Tyr; Tyr into Trp; and/or Phe into Val, into Ile or into Leu.

In another embodiment, the disclosure provides fusion proteins, comprising the polypeptide of any embodiment herein fused to a further functional domain. The polypeptides and peptide domains of the invention may include additional residues at the N-terminus, C-terminus, or both that are not present in the polypeptides or peptide domains of the disclosure; these additional residues are not included in determining the percent identity of the polypeptides or peptide domains of the disclosure relative to the reference polypeptide. Such residues may be any residues suitable for an intended use, including but not limited to detection tags (i.e.: fluorescent proteins, antibody epitope tags, etc.), adaptors, ligands suitable for purposes of purification (His tags, etc.), a protein antigen, a diagnostic molecule, compound or protein; a therapeutic compound or protein, or other peptide domains that add functionality to the polypeptides, etc.

The disclosure provides nucleic acids encoding the polypeptide of any embodiment or combination of embodiments of the disclosure. The nucleic acid sequence may comprise single stranded or double stranded RNA (such as an mRNA) or DNA in genomic or cDNA form, or DNA-RNA hybrids, each of which may include chemically or biochemically modified, non-natural, or derivatized nucleotide bases. Such nucleic acid sequences may comprise additional sequences useful for promoting expression and/or purification of the encoded polypeptide, including but not limited to polyA sequences, modified Kozak sequences, and sequences encoding epitope tags, export signals, and secretory signals, nuclear localization signals, and plasma membrane localization signals. It will be apparent to those of skill in the art, based on the teachings herein, what nucleic acid sequences will encode the polypeptides of the disclosure.

In a further aspect, the disclosure provides expression vectors comprising the nucleic acid of any aspect of the disclosure operatively linked to a suitable control sequence. “Expression vector” includes vectors that operatively link a nucleic acid coding region or gene to any control sequences capable of effecting expression of the gene product. “Control sequences” operably linked to the nucleic acid sequences of the disclosure are nucleic acid sequences capable of effecting the expression of the nucleic acid molecules. The control sequences need not be contiguous with the nucleic acid sequences, so long as they function to direct the expression thereof. Thus, for example, intervening untranslated yet transcribed sequences can be present between a promoter sequence and the nucleic acid sequences and the promoter sequence can still be considered “operably linked” to the coding sequence. Other such control sequences include, but are not limited to, polyadenylation signals, termination signals, and ribosome binding sites. Such expression vectors can be of any type, including but not limited plasmid and viral-based expression vectors. The control sequence used to drive expression of the disclosed nucleic acid sequences in a mammalian system may be constitutive (driven by any of a variety of promoters, including but not limited to, CMV, SV40, RSV, actin, EF) or inducible (driven by any of a number of inducible promoters including, but not limited to, tetracycline, ecdysone, steroid-responsive). The expression vector must be replicable in the host organisms either as an episome or by integration into host chromosomal DNA. In various embodiments, the expression vector may comprise a plasmid, viral-based vector, or any other suitable expression vector.

In another aspect, the disclosure provides host cells that comprise the polypeptides, nucleic acids, expression vectors (i.e.: episomal or chromosomally integrated) of any embodiment disclosed herein, wherein the host cells can be either prokaryotic or eukaryotic. The cells can be transiently or stably engineered to incorporate the nucleic acids or expression vector of the disclosure, using techniques including but not limited to bacterial transformations, calcium phosphate co-precipitation, electroporation, or liposome mediated-, DEAE dextran mediated-, polycationic mediated-, or viral mediated transfection.

In another aspect, the disclosure provides methods for treating cancer, comprising administering to a subject in need thereof an amount effective to treat the cancer of a polypeptide comprising an amino acid sequence at least 75%, 80%, 85%, 90%, 91%, 92%, 93%, 94%, 95%, 96%, 97%, 98%, 99%, or 100% identical to the amino acid sequence of SEQ ID NO:15 or 22, wherein the polypeptide binds to TrkA or PD-L1, respectively. As disclosed above, these polypeptides can be used to treat cancer, including but not limited to cancer characterized by tumors that express PD-L1 and/or TrkA.

In a further aspect, the disclosure provides method for generating an immune response in a subject, comprising administering to a subject in need thereof an amount effective to generate an immune response in the subject of a polypeptide a polypeptide comprising an amino acid sequence at least 75%, 80%, 85%, 90%, 91%, 92%, 93%, 94%, 95%, 96%, 97%, 98%, 99%, or 100% identical to the amino acid sequence of SEQ ID NO:11-14, wherein the polypeptide binds to RSV F protein. As disclosed above, these polypeptides can be used to generate an immune response in a subject in need thereof, including but not limited to a subject at risk or exposure to RSV, or a subject having an RSV infection. In some embodiments, the administering elicits an immune response in the subject, such that the subject is protected against infection by RSV. In some embodiments, the methods limit development of an RSV infection. As used herein, “limiting development” includes, but is not limited to accomplishing one or more of the following: (a) generating an immune response (antibody and/or cell-based) to RSV in the subject; (b) generating neutralizing antibodies against RSV in the subject (b) limiting build-up of RSV titer in the subject after exposure to RSV; and/or (c) limiting or preventing development of RSV symptoms after infection.

In each of these aspects, an “effective amount” refers to an amount of the polypeptide that is effective for treating cancer or generating an immune response.

The polypeptide are typically formulated as a pharmaceutical composition (in combination with a pharmaceutically acceptable carrier), and can be administered via any suitable route, including orally, parentally, by inhalation spray, rectally, or topically in dosage unit formulations containing conventional pharmaceutically acceptable carriers, adjuvants, and vehicles. The term parenteral as used herein includes, subcutaneous, intravenous, intra-arterial, intramuscular, intrasternal, intratendinous, intraspinal, intracranial, intrathoracic, infusion techniques or intraperitoneally. Polypeptide compositions may also be administered via microspheres, liposomes, immune-stimulating complexes (ISCOMs), or other microparticulate delivery systems or sustained release formulations introduced into suitable tissues (such as blood). Dosage regimens can be adjusted to provide the optimum desired response (e.g., a therapeutic or prophylactic response). A suitable dosage range may, for instance, be 0.1 μg/kg-100 mg/kg body weight of the polypeptide or nanoparticle thereof. The composition can be delivered in a single bolus, or may be administered more than once (e.g., 2, 3, 4, 5, or more times) as determined by attending medical personnel.

The subject may be any subject having cancer or at risk of RSV infection. In one embodiment, the subject is a mammalian subject. In another embodiment, the subject is a human subject.

In one aspect, the disclosure provides calcium imaging probes, comprising a polypeptide comprising an amino acid sequence at least 75%, 80%, 85%, 90%, 91%, 92%, 93%, 94%, 95%, 96%, 97%, 98%, 99%, or 100% identical to the amino acid sequence selected from SEQ ID NO:9-10, wherein the polypeptide binds to Ca²⁺. As disclosed above, these polypeptides may be used, for example, for calcium detection.

In one aspect, the disclosure provides electron microscopy stains, comprising a polypeptide comprising an amino acid sequence at least 75%, 80%, 85%, 90%, 91%, 92%, 93%, 94%, 95%, 96%, 97%, 98%, 99%, or 100% identical to the amino acid sequence selected from SEQ ID NO:1-8, wherein the polypeptide binds to Co²⁺. As disclosed above, these polypeptides may be used, for example, to bind to heavy metals such as Co²⁺, in staining a sample for electron microscopy.

In another aspect, the disclosure provides methods for protein design comprising: accessing amino acid sequences and structures of reference proteins;

training a computational model, using the amino acid sequences, the structures, and fragments thereof, to perform functions comprising:
receiving data comprising an amino acid sequence and/or a structure of a functional site or a portion thereof; and
generating an inferred amino acid sequence and/or an inferred structure that scaffolds the functional site and maintains the amino acid sequence and the structure of the functional site.

As described in the examples, the methods of the disclosure (referred to as “inpainting” or “missing information recovery” approaches) start from a desired functional site and jointly fill in the missing sequence and structure needed to complete the protein (i.e.: functional site scaffolding). As used herein, inpainting means predicting the sequence and structure of a missing region of a protein. In some examples, the functional site can be defined by a few side chains around e.g. a metal site. In other examples, a few amino acids on either side of the key residues are also considered to be part of the functional site. For example, in the di-iron case, although there are only 6 residues that contact the metals, about 30 residues can be considered part of the functional site.

In one embodiment, the structures of the reference proteins characterize bonds between amino acids of the reference proteins. In another embodiment, training the computational model comprises minimizing an overall difference between the reference proteins and proteins that are inferred by the computational model when the computational model is provided the fragments of the amino acid structures and/or structures of the reference proteins. In a further embodiment, the structures of the reference proteins include one or more of secondary structure, tertiary structure, or quaternary structure of the reference proteins.

In one embodiment, the reference proteins comprise a particular reference protein, wherein the fragments of the reference proteins comprise a fragment of the particular reference protein, and wherein training the computational model comprises:

generating an inferred fragment that together with the fragment of the particular protein defines an inferred protein, wherein the inferred fragment includes only amino acid sequence information; and

comparing the inferred protein to the particular reference protein.

In another embodiment, the reference proteins comprise a particular reference protein, wherein the fragments of the reference proteins comprise a fragment of the particular reference protein, and wherein training the computational model comprises:

generating an inferred fragment that together with the fragment of the particular protein defines an inferred protein, wherein the inferred fragment includes amino acid sequence information and structural information; and

comparing the inferred protein to the particular reference protein.

In a further embodiment, the reference proteins comprise a particular reference protein, wherein the fragments of the reference proteins comprise a fragment of the particular reference protein, and wherein training the computational model comprises:

generating an inferred fragment that together with the fragment of the particular protein defines an inferred protein, wherein the inferred fragment includes only structural information; and

comparing the inferred protein to the particular reference protein.

In a still further embodiment, the reference proteins comprise a first reference protein, a second reference protein, and a third reference protein, wherein the fragments of the reference proteins comprise a first fragment of the first reference protein, a second fragment of the second reference protein, and a third fragment of the third reference protein, and wherein training the computational model comprises:

(a) randomly selecting a task from a group that includes a first training task, a second training task, and a third training task;

(b) performing the task; and repeating steps (a) and (b) one or more times, wherein:
the first training task comprises:
   generating a first inferred fragment that together with the first fragment defines a first inferred protein, wherein the first inferred fragment includes only amino acid sequence information; and
   comparing the first inferred protein to the first reference protein, the second training task comprises:
   generating a second inferred fragment that together with the second fragment defines a second inferred protein, wherein the second inferred fragment includes amino acid sequence information and structural information; and
   comparing the second inferred protein to the second reference protein, and the third training task comprises:
   generating a third inferred fragment that together with the third fragment defines a third inferred protein, wherein the third inferred fragment includes only structural information; and
   comparing the third inferred protein to the third reference protein.

In one embodiment, the fragments of the reference proteins respectively include at least 15% of the amino acid sequences of the reference proteins. In another embodiment, the fragments of the reference proteins respectively include at least 15% of the structures of the reference proteins. In a further embodiment embodiment, the method further comprises randomly selecting amino acid sequences that make up the fragments of the reference proteins.

In one embodiment, the method further comprises randomly selecting the structures that make up the fragments of the reference proteins. In another embodiment, the fragments of the reference proteins each include at least 10 amino acids. In a further embodiment, the fragments of the reference proteins each include no more than 35 amino acids. In a still further embodiment, the data does not include amino acid sequence or structure other than for the functional site.

In one embodiment, the disclosure provides protein design methods, comprising receiving data comprising an amino acid sequence and/or a structure of a functional site or a portion thereof; and generating an inferred amino acid sequence and/or an inferred structure that scaffolds the functional site and maintains the amino acid sequence and the structure of the functional site. In one embodiment, the structure of the functional site characterizes bonds between amino acids of the functional site. In a further embodiment, the inferred structure characterizes bonds between amino acids of the inferred amino acid sequence.

In one embodiment, the inferred amino acid sequence is a first inferred amino acid sequence, and the inferred structure is a first inferred structure, the method further comprising:

generating a second inferred amino acid sequence and/or a second inferred structure that scaffolds the functional site and maintains the amino acid sequence and the structure of the functional site; and

determining whether (a) the first amino acid sequence and/or the first inferred structure or (b) the second amino acid sequence and/or the second inferred structure better conforms to one or more functional criteria.

In one embodiment, the data does not include amino acid sequence or structure other than for the functional site.

The disclosure also provides polypeptides designed by the protein design method of any embodiment or combination of embodiments herein.

The disclosure also provides non-transitory computer readable media storing instructions that, when executed by a computing device, cause the computing device to perform the method of any embodiment or combination of embodiments of the protein design methods herein. The non-transitory computer readable medium can be any type of memory, such as volatile memory like random access memory (RAM), dynamic random access memory (DRAM), static random access memory (SRAM), or non-volatile memory like read-only memory (ROM), flash memory, magnetic or optical disks, or compact-disc read-only memory (CD-ROM), among other devices used to store data or programs on a temporary or permanent basis. Additionally, the non-transitory computer readable medium can store instructions. The instructions are executable by the one or more processors to cause the computing device to perform any of the functions or methods described herein.

In another embodiment, the disclosure provide computing devices comprising:

one or more processors; and

a computer readable medium storing instructions that, when executed by the one or more processors, cause the computing device to perform the method of any embodiment or combination of embodiments of the protein design methods herein.

The computing device includes one or more processors, a non-transitory computer readable medium, a communication interface, and a user interface. Components of the computing device are linked together by a system bus, network, or other connection mechanism. The one or more processors can be any type of processor(s), such as a microprocessor, a field programmable gate array, a digital signal processor, a multicore processor, etc., coupled to the non-transitory computer readable medium.

The communication interface can include hardware to enable communication within the computing device and/or between the computing device and one or more other devices. The hardware can include any type of input and/or output interfaces, a universal serial bus (USB), PCI Express, transmitters, receivers, and antennas, for example. The communication interface can be configured to facilitate communication with one or more other devices, in accordance with one or more wired or wireless communication protocols. For example, the communication interface can be configured to facilitate wireless data communication for the computing device according to one or more wireless communication standards, such as one or more Institute of Electrical and Electronics Engineers (IEEE) 801.11 standards, ZigBee standards, Bluetooth standards, etc. As another example, the communication interface can be configured to facilitate wired data communication with one or more other devices. The communication interface can also include analog-to-digital converters (ADCs) or digital-to-analog converters (DACs) that the computing device can use to control various components of the computing device or external devices.

The user interface can include any type of display component configured to display data. As one example, the user interface can include a touchscreen display. As another example, the user interface can include a flat-panel display, such as a liquid-crystal display (LCD) or a light-emitting diode (LED) display. The user interface can include one or more pieces of hardware used to provide data and control signals to the computing device. For instance, the user interface can include a mouse or a pointing device, a keyboard or a keypad, a microphone, a touchpad, or a touchscreen, among other possible types of user input devices. Generally, the user interface can enable an operator to interact with a graphical user interface (GUI) provided by the computing device (e.g., displayed by the user interface).

The description of embodiments of the disclosure is not intended to be exhaustive or to limit the disclosure to the precise form disclosed. While the specific embodiments of, and examples for, the disclosure are described herein for illustrative purposes, various equivalent modifications are possible within the scope of the disclosure, as those skilled in the relevant art will recognize.

### Examples

Abstract Current approaches to de novo design of proteins harboring a desired binding or catalytic motif require pre-specification of an overall fold or secondary structure composition, and hence considerable trial and error can be required to identify protein structures capable of scaffolding an arbitrary functional site. Here we describe two complementary approaches to the general functional site scaffolding problem that employ the RosettaFold™ and AlphaFold™ neural networks which map input sequences to predicted structures. In the “inpainting” or “missing information recovery” approach, we start from the desired functional site and jointly fill in the missing sequence and structure needed to complete the protein in a single forward pass through a modified RosettaFold™ network. Inpainting offers greater computational efficiency and accuracy in some regimes. We illustrate the application of the new methods to the design of candidate immunogens presenting epitopes recognized by neutralizing antibodies, receptor traps for escape-resistant viral inhibition, metalloproteins and enzymes, and protein binding proteins. AlphaFold™ structure predictions suggest the designed sequences fold to the designed structures, and neutralizing antibody, metal ion, and protein target binding assays on designs expressed in bacteria confirm the designed functions.

The biochemical functions of proteins are often carried out by a subset of residues which constitute a functional site—for example, an enzyme active site or a protein or small molecule binding site—and hence the design of proteins with new functions can be divided into two steps. The first step is to identify functional site geometries and amino acid identities which produce the desired activity—this can be done using quantum chemistry calculations in the enzyme case (to identify ideal theozymes for catalyzing a desired reaction) (1-3) or fragment docking calculations in the protein binder case (4, 5); alternatively functional sites can be extracted from a native protein having the desired activity (6, 7). In this paper, we focus on the second step: given a functional site description from any source, design an amino acid sequence which folds up to a three dimensional structure containing the site. Current methods have the limitations that assumptions must be made about the secondary structure of the scaffold, and that the amino acid sequence must be generated in a subsequent sequence step, so there is no guarantee that the generated backbones are in fact designable (encodable by some amino acid sequence). An ideal method for functional de novo protein design would 1) embed the functional site with minimal distortion in a designable scaffold protein; 2) be applicable to arbitrary site geometries, searching over all possible scaffold topologies and secondary structure compositions for those optimal for harboring the specified site, and 3) jointly generate backbone structure and amino acid sequence.

### Generalized Functional Motif Scaffolding by Missing Information Recovery

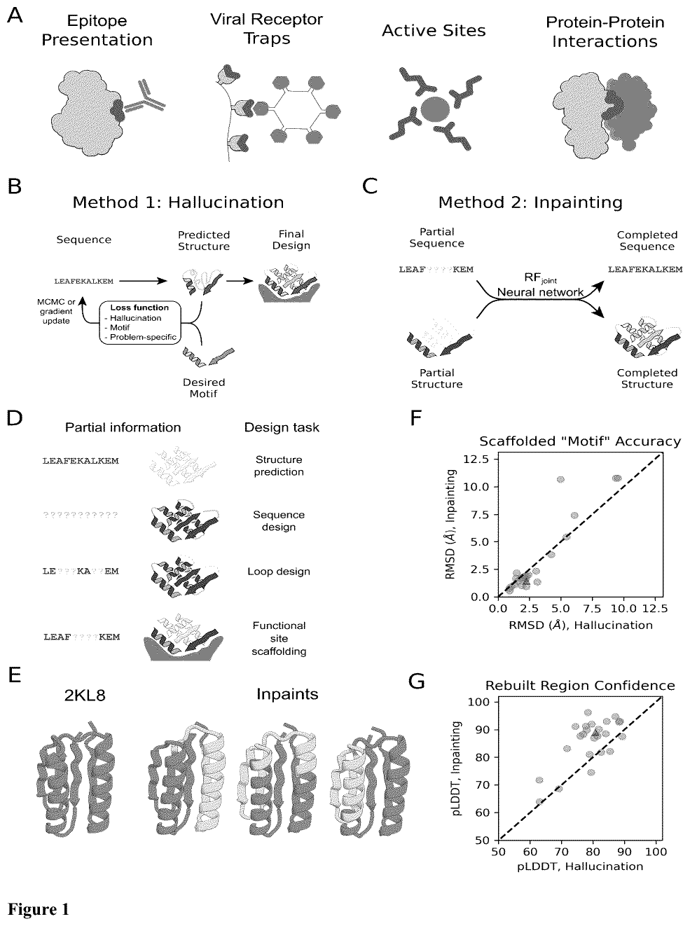

In the training of AlphaFold™ and recent versions of RosettaFold™ (see methods) for structure prediction a small fraction (15%) of tokens in the MSA are masked or corrupted, and the network learns to recover this missing sequence information in addition to predicting structure. We reasoned that this ability to recover sequence information along with structural information could provide a second solution to the functional site scaffolding problem: given a functional site description, a forward pass through the network could potentially be used to complete, or “inpaint”, both protein sequence and structure in a missing/masked region of protein (FIG. 1C; Methods). Here, the design challenge is formulated as an information recovery problem, analogous to the completion of a sentence given its first few words using language models (16) and completion of corrupted images using inpainting methods (17). As illustrated in FIG. 1D, a wide variety of protein structure prediction and design challenges can be similarly formulated as missing information recovery problems.

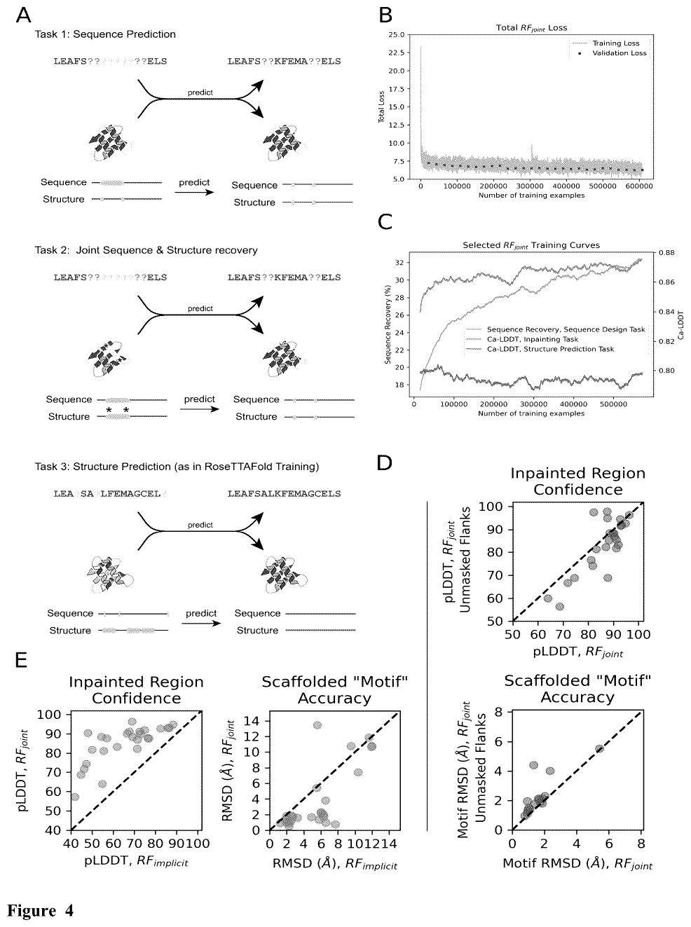

To test whether improving ability of RosettaFold™ to predict missing sequence might help it to better simultaneously reason over both sequence and structure (i.e. to inpaint), we began from a RosettaFold™ model trained for structure prediction (15) and carried out further training on fixed-backbone sequence design in addition to the standard fixed-sequence structure prediction task (Materials and Methods). Despite not being explicitly trained on inpainting (predicting the sequence and structure of a missing region of protein), this model, denoted 1 RF_{implicit}, was indeed able to recover small, contiguous regions missing both sequence and structure. Encouraged by this initial result, we trained a model explicitly on inpainting segments with missing sequence and structure given the surrounding protein context, in addition to sequence design and structure prediction tasks (FIG. 4A; Materials and Methods). The resulting model was able to inpaint missing regions with high fidelity (FIGS. 1E, 4) and performed well at sequence design (32% native sequence recovery during training, FIG. 4C), while maintaining good structure prediction accuracy (FIG. 4C). We refer to this network as RFⱼₒᵢₙₜ and used it to generate all inpainted designs below unless otherwise noted.

To evaluate in silico the quality of designs generated by our methods, we use the AlphaFold™ (AF) protein structure prediction network (18) which has high accuracy on de novo designed proteins (19). Although RosettaFold™ and AF share architectural similarities and were both trained on structures from the Protein Data Bank, the two models were trained independently and hence AF predictions can be regarded as a partially orthogonal in silico test of whether RF designed sequences fold into the intended structures, analogous to traditional ab initio folding benchmarks (13, 20).

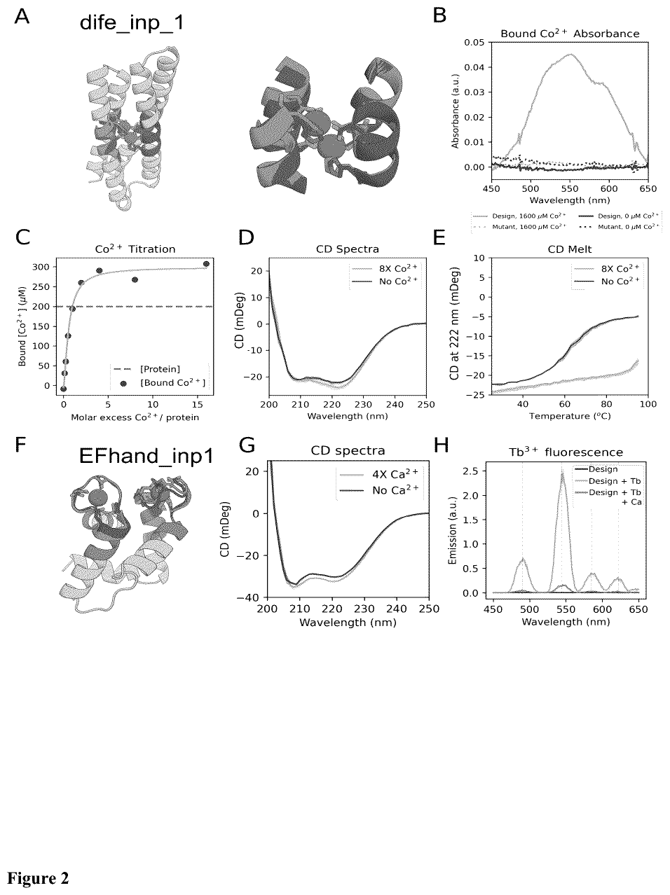

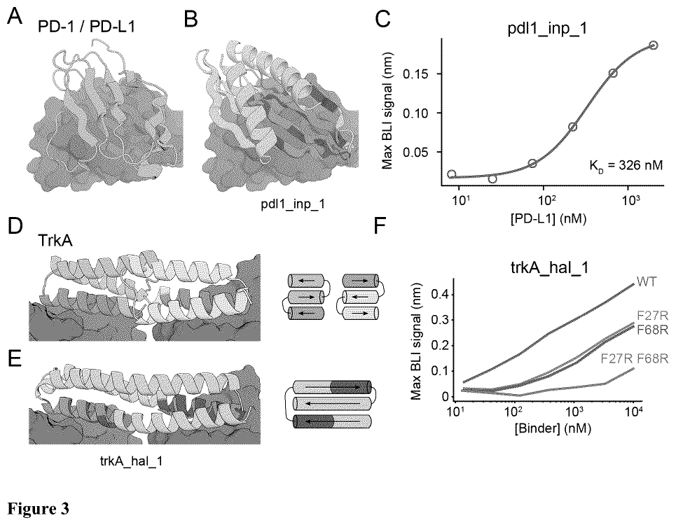

In the following sections, we highlight the power of the inpainting methods by designing proteins containing a wide range of functional motifs (FIG. 2-3, Table 2). For almost all problems, we obtained designs that are closely recapitulated by AF with overall and motif RMSD typically <2 Å and <1 Å respectively with model confidence pLDDT>80 (Table 3). We note that despite their accuracy on previous de novo proteins, AF predictions do not always correlate with protein stability (21) or mutational effects (22), and therefore for most problems below we also validate our designs experimentally (the remaining designs are labeled “in silico” in the corresponding figure panels).

### Designing Metal-Coordinating Proteins

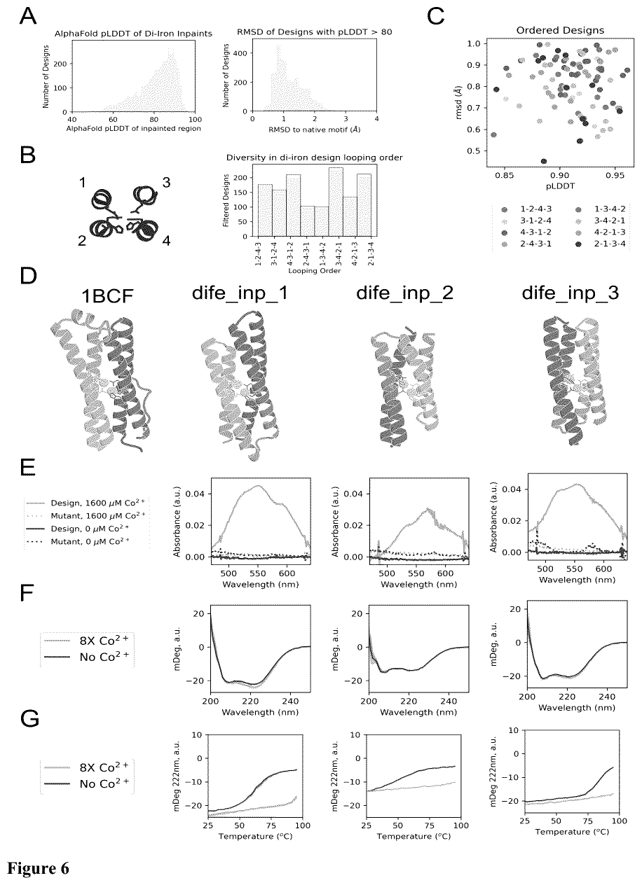

We next explored the scaffolding of functional sites involved in metal-binding and catalysis. We designed scaffolds around a di-iron binding site, which is important in biological systems for iron storage (25) and also potentially harnessable for catalysis (26, 27). The motif, composed of four roughly parallel helical segments from E. co/i bacterioferritin (cytochrome b1), was recapitulated with sub-angstrom RMSDs with inpainting (FIGS. 2A-E, 6). The designs had different helix connectivities (FIG. 6B) than the parent, and are structurally distinct from it (TM-score 0.55-0.71 to 1BCF_A). Inpainting generated a diverse set of 4-helix bundles scaffolding the di-iron binding site without extraneous buried polar residues (FIG. 6). We chose 96 inpainted designs for experimental testing (Supplementary Text). Of these, 76 designs showed clear soluble expression, and for 12 of these we assessed metal binding by measuring the spectroscopic shift in Co²⁺ absorbance, as has been done with bacterioferritin and other designed proteins (28, 29). 8 of the 12 designs displayed a spectroscopic shift at wavelengths consistent with coordination of Co² when incubated with CoCl₂. We characterized 3 of these with the clearest Co²⁺-binding spectra (dife_inp_1-3, FIG. 2B, 6E) in greater depth. Their CD spectra were consistent with the designed fold (FIG. 2D, 6F) and showed significant stabilization upon metal binding to the motifs, as assessed by thermal melt experiments (FIG. 2E, 6G). Mutation of the six iron-binding residues (shown in bold font in Table 1, SEQ ID NO:1-8) to alanine abolished the binding to CO²⁺ (FIG. 2B, 6E). Finally, titration analysis of dife_inp_1 suggested that both metal binding sites were successfully scaffolded (FIG. 2C).

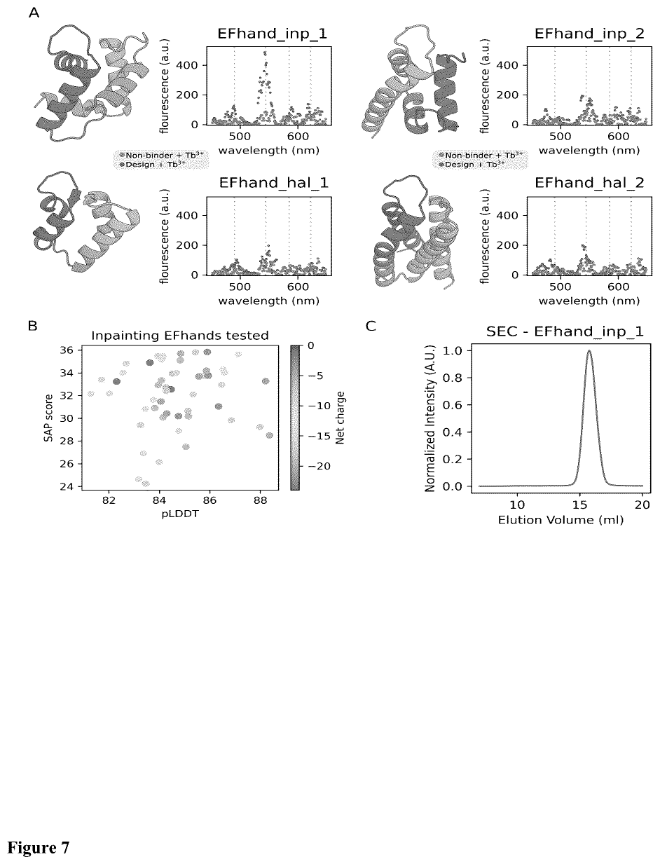

We next pursued scaffolding the calcium-binding EF-hand motif (30) composed of a 12-residue loop flanked by helices. Inpainting readily generated scaffolds recapitulating either 1 or 2 EF-hand motifs to within 1.0 Å RMSD of the native motif (FIG. 2F, FIG. 7A,B, table 3). We chose 20 hallucinations and 55 inpaints to display on yeast and screen for calcium binding using tryptophan-enhanced terbium fluorescence (31). 4 inpaintings had above-background fluorescence at wavelengths consistent with ion binding (FIG. 74A, Materials and Methods; one of these proteins (EFhand_inp_2) was designed using RF_{implicit}). The top hit from yeast, the inpainted EFhand_inp_1, was purified from E. coli as a monomer (FIG. 7C), had the expected CD spectrum (FIG. 2G) and a clear terbium binding signal (FIG. 2H). Competition experiments demonstrated that terbium fluorescence could be reduced by addition of CaCl₂) (FIG. 2H), indicating that the terbium was indeed binding to Ca²⁺-binding sites.

### Designing Protein-Binding Proteins

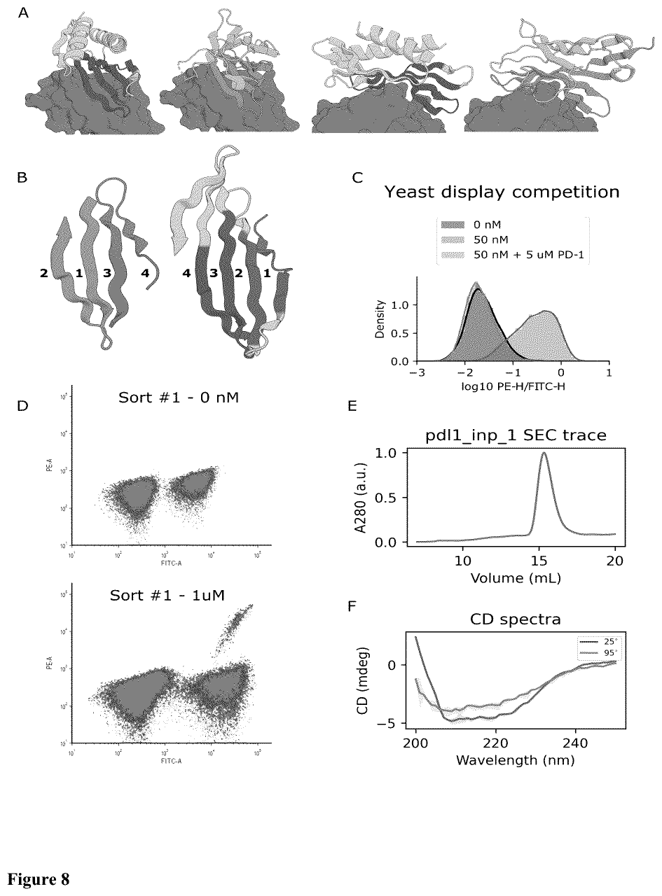

We next explored the design of protein-binding proteins by scaffolding short segments from known binding proteins. To design binders to the cancer checkpoint protein PD-L1, we scaffolded 2 discontiguous segments of the interfacial beta-sheet from a high-affinity mutant of PD-1 (FIG. 3A; Methods) (36). Applying inpainting to this problem yielded a number of designs that not only have good AF predictions of the binder monomer (AF pLDDT>80, motif RMSD<1.4 Å) but also of the complex between the binder and PD-L1, with an inter-chain predicted alignment error (inter-PAE) of <10 Å(Materials and Methods). It was not necessary to redesign the inpainted sequences using Rosetta™. Of 31 designs selected for experimental testing, one design, pdl1_inp_1, bound PD-L1 with a K_{D} of 326 nM (FIG. 3B-C), worse than HAC PD-1 (K_{D}=110 μM) (37) but better than WT PD-1 (K_{D}=3.9 μM) (37). pdl1_inp_1 expressed as a monomer (FIG. 8E), was quite thermostable and had a CD spectrum consistent with that of a mixed alpha-beta fold (FIG. 8F). Unlike native PD-1, which has a immunoglobulin family beta-sandwich fold, pdl1_inp_1 has 2 helices buttressing the interfacial beta sheet, as well as an additional 5th, inpainted beta strand extending the interface (FIG. 8A,B). The closest PDB and BLAST NR hits had a TM-score 0.61 and sequence identity 25.4%, respectively.

The cell surface receptor TrkA dimerizes upon binding to its ligand, nerve growth factor (NGF) (38). Previously, a de novo three-helix bundle was designed to bind TrkA and found to antagonize downstream signaling (4). With the goal of making bivalent TrkA binders that could stimulate signaling, we aligned two copies of the previous TrkA binder three-helix bundle to the signaling-competent TrkA structure (FIG. 3D) scaffolded the two separate hydrophobic binding helices onto a single chain. Following diversification by inpainting (Materials and Methods), one design was predicted to be well-structured (AF pLDDT>80) and interact with TrkA (inter-PAE<10 Å for at least one binding site). It was expressed, purified and found to bind TrkA as assessed by biolayer interferometry (BLI). A double mutant that knocked out both designed binding sites abolished TrkA binding, while single mutants knocking out either one of the binding sites maintained partial binding (FIG. 3F), suggesting that the protein binds two molecules of TrkA as designed.

### Materials and Methods

Training RosettaFold™ to jointly model sequence and structure (RFⱼₒᵢₙₜ₎Standard RosettaFold™ (15) (RF) has been trained on structure prediction (sequence inputs, structure outputs) using homolog templates (structure input). In the newer versions, we mask a portion of the input MSA and apply a loss to predictions of the masked amino acids (sequence output) to encourage the network to extract more meaning from the MSA (18, 65). RFⱼₒᵢₙₜ was fine tuned from a pre-trained RosettaFold™ model (RF-Nov05-2021, see Supplementary Text, “RosettaFold™ variants” section for details on the architectural details of this model). The training regime for this model, which was initially trained solely on structure prediction, is below:

Training set: 25% of examples came from the PDB (published before February 17th, 2020), which is the same training set used in the original RosettaFold™ model (15). The other 75% of examples included a distillation set of AlphaFold™ predicted structures (66). This distillation set was clustered at 30% sequence identity cutoff, and sequences sharing greater than 30% similarity to any protein in the PDB were excluded. Only proteins greater than 200 residues in length, with mean AlphaFold™ pLDDT>85 were included in training, and only residues with per-residue pLDDT>70 were included from these models. The Adam W Optimizer was used throughout training, with default pytorch parameters. The epoch size was 25600 training examples, with a batch size of 64. The learning rate for the initial round of training (200 epochs) was 0.001, with a linear warm-up for the first 1000 optimization steps. The learning rate was then decayed by a factor of 0.95 after every 10000 optimization steps. A crop size of 256 residues was used, with cropping following the same strategy as described previously (15). The number of MSA seed sequences was 128, and the number of extra MSA sequences was 1024. For the second stage of training (100 epochs), the learning rate was set of 0.0005 (no warm-up), with learning rate decay by a factor of 0.95 every 10000 optimization steps. A larger crop size (350 residues), and more MSA sequences (256 seed sequences, 2048 extra sequences) were used in this second phase of training.

Starting with this pre-trained RosettaFold™, we fine-tuned this model for inpainting, for an additional 27 epochs on three tasks (FIG. 4), training only on the PDB training set. For tasks 1 and 2 (fixed backbone sequence design, and inpainting respectively, chosen 33% of the time each) were masked in essentially the same manner. Contiguous regions of 10-35 amino acids comprising at least one full secondary structure element (helix, loop or strand) were masked out (Task 1: only sequence masked; Task 2: sequence and structure masked). The sequence and structure of a further 3-6 ‘flanking’ residues were masked out either side of this contiguous region (FIG. 4A). The distograms (but not angle maps or amino acid identity) were provided for the residue immediately N- and C-terminal to the central contiguous masked region (FIG. 4A, asterisks). Noise was also applied to these two positions, by randomly translating them following a normal distribution (=0 Å, σ=1 Å), such that at inference time, coordinates would be provided to the network as a “guide” rather than as absolute positions. Losses were not applied to the flanking regions either side of these two coordinates. The masking of flanking sequence and structure modestly improved the performance of the network in the benchmarking test, compared to just masking a 10-35 residue window (FIG. 4D). The final task (structure prediction from MSA information) was the original task the pre-trained RosettaFold™ was trained on, which differs slightly from the original RosettaFold™ network (15). Specifically, in this task, 15% of the MSA (excluding the input sequence) was randomly masked or corrupted (following the strategy used by AlphaFold™ (18), of this 15% of residues, 70% of residues were replaced with a ‘mask’ token, 10% were mutated to a random amino acid, 10% were mutated to another amino acid in the MSA column, and 10% were not replaced). Homologous template structural inputs were unchanged from the original network (15). The applied loss function was the same for all three tasks:

The loss function formulation for RFⱼₒᵢₙₜ is as follows.

[symbol]

ₜₒₜₐₗ=1.0 [symbol] _{dist}+3.0 [symbol] ₐₐ+1.0 [symbol] ₜₒᵣₛ+5.0 [symbol] _{FAPE}+0.1 [symbol] _{lddt}

where [symbol] _{dist} is a cross entropy loss over the distogram and anglegram as described in (15), predictions [symbol] ₐₐ is a cross entropy loss over any masked positions in the input MSA, [symbol] ₜₒᵣₛ is a cross entropy loss on binned backbone dihedral angle predictions, [symbol] _{FAPE} is a backbone level frame aligned point error, as described in (18), with a relu cutoff of 20. [symbol] _{lddt} is the 1DDT loss as calculated in (15). Note that structure related losses are applied over the entire predicted protein, and the sequence cross entropy loss is only applied at masked (Tasks 1 and 2) and/or corrupted (Task 3) regions. For the fixed-backbone sequence design task (FIG. 4A, Task 1) and for the inpainting task (FIG. 4A, Task 2), no loss was applied on the ‘flanking’ region of protein N- and C-terminal to the central masked region. The learning rate was set to 0.0003 throughout the training of these three tasks, with a batch size of 512. We refer to this fine-tuned RosettaFold™ inpainting model as RF_{Joint}, and selected training curves from this model are shown in FIG. 4B,C. Details of a different training strategy used to train an earlier version of the inpainting network, which implicitly learned to inpaint, are provided in the supplementary methods.

Joint Sequence-Structure Inpainting with a Jointly Trained RosettaFold™

To apply RFⱼₒᵢₙₜ to protein design, we input a sequence and structure, masking certain residues in the sequence by replacing them with mask tokens and masking corresponding residues in the structure by setting their template embeddings to zero (15). We then predict the structure and sequence logits for the entire protein. The output structure, including regions that were originally both masked and unmasked, is used as the design model, and the most probable predicted amino acid at each masked position (argmax) is taken to complete the sequence. Note that in the RF-Nov05-2021 version of RosettaFold™ used to train RFⱼₒᵢₙₜ, as in AlphaFold™, latent representations of the output structure are ‘recycled’ back through the network to refine the final structure. During inpainting, we utilize this ‘recycling’ to refine our inpainted sequence and structure, typically recycling information 5-15 times (similar to the number of times used for structure prediction with RosettaFold™, which is typically 10). A single design of 100 amino acids in length, using 10 iterations of inpainting, takes 5.3 seconds on a GeForce RTX 2080 GPU. We refer to this prediction, with recycling, as a ‘forward pass’ through the network. The iterative inpainting method described above is approximately deterministic. To sample ensembles of outputs with small variations in sequence and structure using RFⱼₒᵢₙₜ, we either vary the exact boundaries of masked regions, the length of regions to replace a masked region or by varying specific input coordinates (for example, in FIG. 56C, the coordinates of two Cα-coordinates were randomly translated up to a specified distance from their original positions, and the network was tasked with inpainting the masked region given the unmasked positions of the two translated residues). For each of the design cases presented in the paper, the precise strategy used to generate and filter the designs is described in the supplementary methods.

### Design Filtering & Selection

For each experimentally tested design case shown in this paper, we generated between 4000 and 30,000 designs, and filtered these based on the AF pLDDT, motif RMSD of AF predictions, (see supplementary text for exact cutoffs). Broadly, these included ‘confident/accurate’ AF pLDDT (>80), sub-angstrom (<1 Å) RMSD. Orthogonal filters were determined on a per-problem basis (fully outlined in the supplementary text), but broadly comprised features such as radius of gyration, Rosetta™ per-residue spatial aggregation propensity (SAP) score (67), net charge (#Arg+#Lys—#Asp—#Glu) and structural diversity. The cutoffs were typically chosen to give an experimentally tractable final number of designs. In some cases, in preparation of the final set of proteins to be ordered, and after design filtering, we performed a final visual inspection to look qualitatively at aspects such as poor core packing, presence of cavities, buried polar groups, or surface hydrophobics, which typically reduced the set of proteins by around 0-50%.

For designs that were only validated in silico, that are represented in the figures, we filtered designs predominantly on AlphaFold™ pLDDT and RMSD, as well as radius of gyration. The AlphaFold™ metrics are presented in Table 3.

The “model 4” weights were used for all AF predictions for filtering. The pLDDT was taken as the average of the residue-wise confidence values output by the network. Using AF to filter our designs has the risk of designing “adversarial examples”, or sequence-structure pairs that score well by AF that do not fold or function in reality, due to the presence of artifactual minima in the loss landscape of the structure-prediction model (68, 69). However, because we design using RosettaFold™, which is trained independently of AF (although both use the PDB as training data), any final designs must be well-predicted by two partially orthogonal networks, which is expected to provide some (although not total (70)) robustness to adversarial examples. This is supported by our finding that a high fraction of our designs are solubly expressed. Additionally, if we redesign the sequence of our highest-pLDDT designs by Rosetta™, pLDDT continues to be high, indicating that the original hallucination had a designable backbone (and isn't purely an artifact of RF or AF's loss landscape). Finally, we find that AF pLDDT of our RF-generated designs correlate well with physics-based metrics such as Rosetta™ energy and ab initio folding.

To score protein binder designs, we used a modified AlphaFold™ prediction script that took as input the design model of the target-binder complex (from inpainting) and the concatenated binder-target sequence (with a residue number gap to denote different chains). AF was asked to predict the complex structure from single-sequence, given the target protein structure as template information and its structural representation (atom coordinates) of the binder-target complex initialized to the target-binder complex design model. The confidence in AF2's prediction of the interface was assessed by the inter-chain predicted aligned error (inter-PAE), or the average value of interchain positions in the predicted aligned error matrix. We found that inter-PAE<10 Å corresponded to predicted complexes that were docked roughly correctly, while predictions with inter-PAE above this threshold usually had binder and target far apart in space. In addition to inter-PAE, we also filtered on: binder pLDDT (average residue-wise confidence over the binder from complex prediction); AF-Rosetta™ ddG (Rosetta™ ddG calculated on the AF model after minimizing interface side chains); target-aligned binder RMSD (RMSD of the binder, after aligning AF and RF models on the target).

### Protein Purification

All designs tested in E. Coli were cloned, expressed and purified using standard methods. Briefly, Golden Gate assembly with BsaI-HF (New England Biolabs) was used to insert designs into a modified pET29b+ vector containing C-terminal SNAC (71) and 6×His tags (or, in the case of EFhand_inp_1, into a modified pET29b+ vector with a C-terminal TEV cleavage site and a 6×His tag). Plasmids were transformed into BL21 bacteria. For small-scale expression tests, bacteria were cultured overnight at 37° C. in 2 ml cultures of lysogeny broth (LB) supplemented with 50 μg/mL of kanamycin. Cells were then grown in 2 ml cultures of Terrific Broth (TB) for one hour, before induction with 1 mM of IPTG for 4 hours. Cells were then lysed with B-PER supplemented with 1 mM PMSF, 0.1 mg/mL Lysozyme, 25 U/ml Benzonase, before lysate clarification by centrifugation. Lysate was incubated with 75 μl Ni-NTA resin, before washing thrice with wash buffer (25 mM Tris, 300 mM NaCl, 20 mM Imidazole, pH 7.8) and elution in 25 mM Tris, 300 mM NaCl, 250 mM Imidazole. Expression was assessed by SDS-PAGE. For larger scale cultures, cultures were grown overnight at 37° C. in autoinduction medium (72), before sonication-based lysis in wash buffer supplemented with 1 mM PMSF, 0.1 mg/mL Lysozyme, 0.01 mg/ml DNase I. After centrifugal lysate clarification, lysates were incubated with an appropriate volume of Ni-NTA resin and subsequently washed thrice with wash buffer. For purification of di-iron binding proteins, the His-tag was cleaved off by cleavage of the SNAC-tag. Briefly, after binding to the Ni-NTA resin, the protein was washed in SNAC cleavage buffer (100 mM CHES, 100 mM Acetone oxime, 100 mM NaCl, 500 mM GuHCl, pH 8.6) before addition of 2 mM NiCl₂. After overnight cleavage, proteins were further purified by size exclusion chromatography on a Superose 75 column in 20 mM Hepes, 100 mM KCl, pH 7.8, and monomeric fractions pooled.

### Spectroscopic Analysis of Cobalt Binding to Di-Iron Binding Proteins

Analysis of cobalt binding to inpainted di-iron binders was performed essentially as described previously (28). Proteins (200 μM in 20 mM Hepes, 100 mM KCl, pH 7.8) were incubated overnight with (or not) an 8×molar excess (1600βM) CoCl₂. Absorbance spectra were collected in a Jason V-750 spectrophotometer. Mean background absorbance (measured between 700 and 800 nm) were subtracted from all spectra. Successful designs showed absorbance peaks characteristic of cobalt coordinated in a tetra/penta-coordinate state.

### Fluorescence Analysis of Terbium Binding to EF-Hand Designs

### Yeast-Displayed Designs

Transformed yeast were cultured in TRP(−), URA(−) media for two days followed by expression culture. Samples containing˜8.5e7 cells were incubated in TBS (pH 8.0) containing 1 mM Ca²⁺ and washed twice with TBS only. Yeast cells were resuspended in TBS containing 50 μM Tb³⁺ For 3 hours and then washed twice in TBS+1 mM Ca²⁺. Washed samples were moved to a black bottom, plate-reader 96 plates for fluorescence spectra measurement. Fluorescence signals were collected using a flash plate reader in time-resolved fluorescence mode (TRF, delay time: 100us, integration time: 1000us, gain: 130).

### Purified Designs

Designs harboring the EF-hand motif, were purified by His-purification as described above. After size exclusion chromatography in 20 mM Hepes, 150 mM KCl, pH 7.8, the His tag was cleaved by TEV-cleavage, with the addition of 40 μM Super-TEV protease, 1 mM DTT and 0.5 mM EDTA (overnight at room temperature). To ensure the EF-hands were not bound to any residual calcium in buffers, after passing through a NiNTA-column after TEV-cleavage, protein were run on a size exclusion column equilibrated in 20 mM Hepes, 150 mM KCl, pH 7.8 buffer, which had been Chelex treated overnight to remove any residual calcium. Proteins were incubated (or not) with terbium (40 μM terbium in 5 μM protein) for 3 hours, before analysis of terbium fluorescence on a NEO2 plate reader. Samples were excited at 250 nm (to excite the tryptophan residue near the EF-hand motif), and fluorescence was measured between 450 and 650 nm, 100-1000 s after excitation.

### Circular Dichroism Spectroscopy

All circular dichroism (CD) analyses except those for RSV-F site V immunogens were performed on a JASCO J-1500 CD Spectrophotometer. Di-iron binding proteins were analyzed at 6.7 μM in 20 mM Hepes, 10 mM KCl, pH 7.8, with or without an 8×molar excess of CoCl₂. Analysis of the EF-hand inpaint was performed at 20 μM in chelex100-treated 20 mM Hepes, 150 mM KF, pH 7.6, in the presence or absence of 200 μM CaCl₂). Analysis of the PDL-1 binder was performed at 5 μM in 20 mM Hepes, 10 mM KCl, pH 7.8. Thermal melt analyses were performed between 25° C. and 95° C., measuring CD at 222 nm. All reported measurements were measured within the linear range of the instrument.

For RSV-F designs, CD spectra were measured using a Chirascan™ V100 spectrometer in a 1-mm path-length cuvette. The protein samples were diluted to 30 μM in PBS. Wavelengths between 195 nm and 250 nm were recorded. Thermal melt analyses were performed between 20° C. and 95° C. with an increment of 2° C./min, measuring CD at 222 nm. All spectra were corrected for buffer absorption.

### Measuring Protein Binding

### Yeast Surface Display

As an initial screen for protein binding, linear DNA were synthesized as “e-blocks” (Integrated DNA Technologies), pooled, and transformed into the yeast strain EBY100 (by electroporation if >100 designs, by the lithium acetate method otherwise) along with a pETCON3 backbone linearized at NdeI and XhoI (for Aga2p and c-Myc fusion) (4, 5). The transformed pool was inoculated into CTUG medium (yeast nitrogen base 6.7 g/L (difco)+complete amino acids−trp−ura+2% glucose) and incubated 12-16 hours at 30° C. with shaking, then diluted 200 uL+2 mL into SGCAA (yeast nitrogen base 6.7 g/L+complete amino acids 5 g/L (Bacto)+90 mM Na₂HPO₄+2% galactose+0.1% glucose) and incubated 12-16 hours to induce binder expression and display. For flow sorting, around 10⁷ cells were harvested, washed 3× in TBSF (50 mM Tris-HCl pH8.0, 150 mM NaCl, 1% bovine serum albumin), incubated in TBSF with biotinylated binding target for 30 minutes at room temperature, washed 1× in TBSF, incubated for 30 minutes at room temperature in 0.1 mg/mL FITC anti-c-Myc (ICL Lab) and 70 mg/mL streptavidin R-phycoerythrin (PE) conjugate (Invitrogen), and washed 3× in TBSF. The binding target and FITC/PE were added in the same incubation when labeling with avidity. Cells were sorted on a Sony SH800 flow sorter and 10³-10⁶ FITC+/PE+cells were collected. The cells were either cultured in liquid CTUG for another round of sorting, or plated onto CTUG agar and individual colonies Sanger-sequenced to identify the designs. For trRosetta™-hallucinated PD-L1 binders and Mdm2 binders, clonal yeast cultures expressing a single design were analyzed in binding assays to confirm the results of sorting as well as to assess the binding affinity of designs. In this case, yeast culture and binding were performed identically as above except that an Attune N×T™ (Invitrogen) flow cytometer was used to analyze the cells. For all other problems, hits identified by yeast display were followed up by E. coli expression and purification.

### Surface Plasmon Resonance (SPR) to Assess RSV-F Site V Binding

SPR measurements were performed on a Biacorem 8K (GE Healthcare) in 10 mM HEPES pH 7.4, 150 mM NaCl, 3 mM EDTA, 0.005% v/v Surfactant P20 (GE Healthcare). Ligands were immobilized on a CM5 chip (GE Healthcare) via amine coupling. The preRSVF and RSVF-site V immunogens were immobilized at approximately 300-500 response units (RU). The site V specific RSV90 Fab was injected as analyte in two-fold serial dilutions. The flow rate was 30 μl/min for a contact time of 120 s followed by 400 s dissociation time. After each injection, the surface was regenerated using 0.1 M glycine at pH 3.0. K_{D} values were obtained by fitting the maximum response versus log 10 Fab concentration to a sigmoid function using Python and scipy's non-linear curve_fit function.

### Biolayer Interferometry (BLI) to Assess Bivalent TrkA Binding

BLI binding experiments were performed on an Octet Red96™ (ForteBio), with streptavidin coated tips (Sartorius Item no. 18-5019) and BLI buffer (10 fold dilution of 10× HBS−EP+buffer [Cytiva Item no. BR100669] supplemented with 0.1% w/v bovine serum albumin). Tips were pre-incubated in BLI buffer for at least 30 minutes before use. To collect binding data, the tips were incubated in BLI buffer for 100 s, loaded with biotinylated TrkA (30 nM in BLI buffer; a kind gift from Chris Garcia's lab) for 300 s, equilibrated in BLI buffer to obtain a baseline for 150 s, dipped into BLI buffer with the designed proteins for 900 s (association phase) and finally returned to BLI buffer for 900 s (dissociation phase). Reported responses are the change in wavelength between the beginning and end of the association phase.

### RosettaFold™ Variants

The protein structure prediction performance of each RosettaFold™ variant was evaluated based on CASP14 targets and 60 recently published de novo designs (not included in the RosettaFold™ training set).

Inpainting models were fine-tuned starting from one of the pre-trained RF versions above. RFⱼₒᵢₙₜ was based on RF-Nov05-2021, and RF_{implicit} was based on RF-perceiver (see dedicated sections for precise training details).

Because RosettaFold™ only predicts backbone coordinates, we added sidechains to inpainting outputs using Rosetta™ and refined the full-atom structure by relaxing once in torsion space with predicted pairwise restraints and once in cartesian space with only pairwise distance restraints and Ca coordinate restraints. The output of the final relax step is the model used for downstream analysis and further design.

### Di-Iron Binding Protein Design by Inpainting

The input motif we sought to scaffold was extracted from bacterial cytochrome b-1 (PDB accession 1BCF), and comprised four approximately parallel helices (residues A18-25, A47-54, A92-99 and A123-130, harboring motif residues Glu^{A18}, Gu^{A51}, His^{A54}, Glu^{A94}, Glu^{A127}, His^{A130}. Eight potential looping orders were inpainted (FIG. 6B), randomly sampling connecting lengths between helices of 16-30 residues, with 8-15 residues inpainted at the N— and C-termini. For each looping order, 500 designs were generated.

While confidently predicted by AlphaFold™ to scaffold the motif, we noticed that some designs had a higher-than-ideal number of surface hydrophobic residues (as assessed by SAP units (67)). Given the ability of RFⱼₒᵢₙₜ to design sequence-given-backbone, for some designs, we used RFⱼₒᵢₙₜ to modestly redesign the sequence to reduce the SAP score. Specifically, we redesigned hydrophobic surface residues to reduce the predicted aggregation propensity (given either the AlphaFold™ or RFⱼₒᵢₙₜ model as backbone-input).

The following filters were used for filtering the inpainted designs:
AlphaFold™ mean pLDDT>80
AlphaFold™ pTM score>0.7
RMSD of AlphaFold™-predicted motif to native<1 Å
Net charge between −25 and −5
Surface hydrophobicity (SAP units)<40 (for designs without surface-redesign) or <34 (for designs with surface redesign)
Rosetta™ Iron-binding energy of at least one site<−2.4

### EF-Hand Design

### Inpainting

The 55 inpainted EF-hand designs tested experimentally contain 51 designs from RFⱼₒᵢₙₜ, and 4 designs from RF_{implicit}.

For RFⱼₒᵢₙₜ designs, we began with 18,000 inpainted designs: 9,000 using native 1PRW as an input template and 9,000 from a version of 1PRW where the backbone is identical but the sequence contains a K30W mutation. In all designs, we combinatorially sampled template inputs that contained
5-20 masked residues at the N terminus, followed by residues A16-35 from 1PRW
10-25 masked residues between A35 and domain A52-71 from 1PRW
5-20 masked residues after A71

We chose to inpaint the second set of 9,000 off of the K30W mutant because the downstream functional assay (tryptophan-enhanced fluorescence) requires tryptophan to be near the ion binding site, and we reasoned that final designs might be higher quality if the model was conditioned on a TRP residue in its input, rather than retrospectively making a TRP mutation on an unconditioned design. The AF2 pLDDT distributions for these two sets of 9,000 designs were nearly identical (mean 77 vs 76), and their motif RMSD distributions were also similar. Given this, we reasoned that a K30W mutation likely would have minimal effect on a design's AF2 prediction metrics (especially given it is a surface mutation in the design). Thus for any designs which passed filters (see below) but were not conditioned on the K30W mutation, we manually added the mutation without further calculation.

We filtered this initial set of 18,000 by AF2 pLDDT>80, and the individual EFhand domain RMSDs both being<1. This yielded 1496 sequences, all of which now had the K30W mutation discussed above. We next created two mutants for each of these sequences to add a second TRP near the binding sites—T26W and F65W (numbering with reference to 1PRW). We then used AF2 to predict the structure of all mutants to ensure the addition of a second TRP was not deleterious for a design's AF2 metrics. Using a filter of AF2 pLDDT<83.7, RMSD of both domains individually<1.0, and SAP score<36, we filtered this set of 2992 designs to the final set of 51 for testing.

For 4 RF_{implicit} designs, we started from two hallucinated designs which initially scaffolded the EFhand motif(s) from 1PRW (Table 3), denoted here as EFhand_hal_A and EFhand_hal_B.

We inpainted 300 designs seeding off of EFhand_ha_A by combinatorially sampling template inputs that contained
5-13 masked residues at the N-terminus, followed by residues A11-30
15-24 masked residues between residue A30 and domain A50-81
4-8 residues after residue A81

We inpainted 300 designs seeding off of EFhand_hal_B by combinatorially sampling template inputs that contained
0-4 masked residues at the N-terminus, followed by residues A7-17
13-28 masked residues between residue A17 and domain A31-55
6-16 masked residues after residue A55

Designs were filtered using AF2 pLDDT>80 and backbone RSMD between the AF2 prediction and the native 1PRW EF-hand on at least one of the motifs. We arbitrarily chose 1 design that passed these filters from each of the two sets of 300 designs. For both proteins, two mutants were created. For both mutants, the K30W (numbering with respect to 1PRW) mutation as seen above was made. Then the T26W mutation was made for one mutant, and the F65 mutant mutation was made for the other. This process yielded 4 tested designs, one of which showed terbium binding activity in the yeast display terbium binding assay (FIG. 7, EFhand_inp_2).

### PD-L1 Binder (PD-1 Mimetic) Design

We used hallucination and inpainting to scaffold a 2-segment beta-sheet motif from the high-affinity consensus (HAC) PD-1 interface toward PD-L1 (5IUS chain A residues 63-82, 119-140) (36) Given the immunoglobulin-like topology of PD-1, these 2 segments do not have nearby N— and C-termini and therefore cannot easily be linked by a short hairpin; therefore, it is non-trivial to scaffold them into any fold other than their native immunoglobulin.

### Inpainting

We generated 2 sets of inpainted designs: “free” inpaintings where only the binding motif was used as input, so RFⱼₒᵢₙₜ would have to generate the entire scaffold from scratch; and “guided” inpaintings where the binding motif, as well as guiding structural information input by hand, were provided. All designs were modeled in the presence of the target PD-L1, analogous to “two-chain” hallucination (Materials and Methods).

For free inpainting, we manually chose a looping order for the design to be inpainted with, starting at the N-terminus with motif segment A1 19-140 from 5IUS, then allowing 22-29 inpainted residues, then segment A63-82 from 5IUS, and finally 28-39 inpainted C-terminal residues. Additionally, we allowed RFⱼₒᵢₙₜ to redesign residues 67, 69, 71, 73, 75, and 77 in the input motif (i.e. mask and re-predict amino-acid identity, taking the most probable amino acid at each position, without masking structure) in case they changed from core to surface, or vice versa, after inpainting. We generated 314 designs using this approach. The successful binder pdl1_inp_1 is a refined (see below) version of a parent design from this set.

For guided inpainting, we tried to bias RFⱼₒᵢₙₜ to explore a topology of a beta-sheet buttressed by 2 helices that was observed in high-scoring hallucinations. To do this we manually placed 5 “guiding” residues in an input structure and asked inpainting to generate a design containing the interface motif which generally goes through the backbone atoms of the guide residues. 4 of the guide residues correspond to the rough location of N and C termini of two helices that might buttress the sheet. The 5th guide residue is placed in the middle of one of the buttressing helices, at an elevated distance above the interfacial beta-sheet so as to induce a bend in the helix to pack against the sheet without clashes. To obtain a diversity of designs, we sampled input coordinates for each guide residue from a uniform random sphere of radius 2 Å around its original manually chosen position, and also combinatorially sampled the lengths of the regions to be inpainted. Specifically, we combinatorially sampled the following template inputs, with each masked region being uniformly sampled from allowed window lengths:
Residues A119-140 from 5IUS
4-6 masked residues between the previous segment and guiding residue 1
12-14 masked residues between guiding residues 1 and 2
4-7 masked residues between guiding residue 2 and A63-82 from 5IUS
5-8 masked residues between the previous segment and guiding residue 3
11-13 masked residues between guiding residues 3 and 4
9-12 masked residues between guiding residues 4 and 5
0-3 masked

Given these inputs, RFⱼₒᵢₙₜ was able to generate a diverse family of PD-1 mimetics with this fold. We generated 1000 parent designs using this approach, although no descendants of these parent designs ended up having binding activity.

After initial design runs, designs with pLDDT>80 and inter-chain PAE<10 were refined using RFⱼₒᵢₙₜ to (1) “resample” the protein by randomly re-inpainting a fraction of the residues, (2) redesign only the sequence (keeping structure) of hydrophobic surface/boundary residues or (3) changing the order in which elements of the protein appear in primary sequence while keeping the overall fold of the protein (“relooping”). Combinations of (1), (2) and (3) were used for exploring near the topology proposed by inpainting initially, as well as optimizing a design for low net charge and low SAP score. We generated a total of 2,025 refinements off of the initial “free inpainting” set and 415 refinements off of the initial “guided inpainting” set, using the AF2 predictions of designs as input backbones for refinement over a maximum of 3 rounds of filtering (pLDDT>80, inter-chain PAE<10) and refinement. The final designs for experimental characterization were redundancy-reduced by mmseqs2 at 90% identity cutoff, and then filtered by Rosetta™ DDG<−30, SAP score<40, net charge<−4, AF2 inter-PAE<10, and AF2 pLDDT>80. This final filtering yielded the pool of 31 tested sequences, one of which bound PD-L1 (FIG. 3A-C).

### Bivalent TrkA Binder Design by Hallucination Followed by Inpainting

We began the design process by aligning the structure of the TrkA minibinder bound to a single domain of TrkA (PDBID: 7N3T) to the complex of TrkA with its native ligand, nerve growth factor (PDBID: 2IFG). Having obtained the relative positions of the two minibinders in a signaling competent TrkA arrangement, we defined the functional motif as residues 5-18 on each of the minibinder chains. We carried out 600 steps of gradient descent with the usual motif and hallucination losses and forcing the native identity on motif residues 5, 6, 9, 10, 12, 13, 14, 16, 17 and 18 from both minibinder chains. To avoid clashes with TrkA, we applied a repulsive loss against the coordinates from the appropriately aligned TrkA structure (σ=3.5 Å, weight=5). Because many of the residues in either of the two motif segments were further from each other than the 20 Å distogram horizon, we also found it necessary to apply a coordinate rmsd loss (weight=1), which has no such distance maximum, to encourage the two motifs to have the correct orientation to each other. The resulting 380 designs were filtered (cce loss<1.0, coordinate rmsd loss<1.5 Å and entropy loss<2.0) down to 9 seed designs. After manual inspection for designs with well-packed secondary structure elements and minimal loops, we chose to diversify one design of an elongated three helix.

To diversify the seed designs, we used inpainting to change the length and position of the two loop regions connecting each helix. First, we made 20 “jittered” structures by adding gaussian noise˜N(0,1) to “guide points” two residues inside each loop region. (Since inpainting is deterministic, this approach allowed us to sample different inpainting solutions for loops of the same length.) For each jittered structure, we inpainted the loops while varying their lengths between −3 and +7 residues of the original length, generating 1280 designs. After filtering for well folded designs (AF pLDDT>80) that interact with TrkA (inter-PAE<10 Å for at least one binding site), one design remained. This design and derivative mutants were assayed for TrkA binding by biolayer interferometry.

TABLE 2
Natural proteins used for mimetic design
“Motif residues” indicate residues that were constrained
to native geometry during hallucination. Sometimes only a
subset of the motif residues actually comprise a binding
interface or catalytic site; these are denoted “functional residues”.

| Native protein | PDB | | | | Binding |
|---|---|---|---|---|---|
| (Reference) | ID | Chain | Motif residues | Functional residues | partner(s) |
| HAC PD-1 (82) | 5IUS | A | A63-82, A119-140 | A64, 66, 68, 70, 73-75, | PD-L1 |
| | | | | 77-78, 81, 85, 89-91, | |
| | | | | 124, 126, 128, 132, 134, | |
| | | | | 136, 139 | |
| RSV-F site II (83) | 3IXT | P | P254-277 | | Antibody |
| RSV-F site V (23) | 5TPN | A | A163-181 | | Antibody |
| ACE2 (84) | 6VW1 | A | A24-42 | | SARS-CoV2 |
| | | | | | receptor |
| | | | | | binding domain |
| EF-hand (85) | 1PRW | A | A21-31, A56-67 | A21-31, A56-67 | Ca²⁺ |
| Di-Fe (25) | 1BCF | A | A18-25, A47-54, | A18, 51, 54, 94, 127, 130 | Fe²⁺ |
| | | | A94-97, A123-130 | | |
| Carbonic | 5YUI | A | A62-65, A93-97, | A94, A96, A119, A199 | Zn²⁺ |
| anhydrase II (32) | | | A118-120 | | |
| Δ⁵-3-ketosteroid | 1QJG | A | A14, A38, A99 | A14, A38, A99 | equilenin |
| isomerase (35) | | | | | |
| p53 N-term helix | 1YCR | B | B17-27 | A19, 23, 26, 27 | Mdm2 |
| (86) | | | | | |
| TrkA minibinder | 7N3T | A | A5-18 | A5, 6, 9, 10, 12, 13, 14, | TrkA |
| (4) | | | | 16, 17, 18 | |

TABLE 3
RMSDs between native protein, design model, and AlphaFold ™ model
All RMSDs are in angstroms. Columns AF pIDDT and RMSD, AF to
native are the metrics reported in the main text and figures. RMSD values
in parentheses (for hcA and KSI) are full-atom RMSDs over the catalytic
sidechains. KSI designs are generated using AF, and “Design” refers
to models generated using the ensembling approach over AF models
1, 2, 3, 5 and “AF” refers to AF model 4 (Materials and methods).

| | Overall | | Motif | | |
|---|---|---|---|---|---|
| | AF | RMSD, | RMSD, | RMSD, Design | RMSD, AF |
| Design | pIDDT | Design to AF | Design to AF | to native | to native |
| rsvfv_hal_1 | 82 | 1.37 | 1.06 | 1.31 | 0.7 |
| rsvfv_hal_2 | 88 | 0.75 | 0.34 | 0.67 | 0.64 |
| rsvfv_hal_3 | 86 | 0.85 | 0.24 | 0.65 | 0.65 |
| rsvf-v_854 | 82 | 2.45 | 0.65 | 0.71 | 0.75 |
| rsv_inp_1 | 83 | 0.91 | 0.5 | 0.51 | 0.59 |
| rsv_inp_2 | 83 | 0.76 | 0.57 | 0.6 | 0.81 |
| rsv_inp_3 | 88 | 1.14 | 0.55 | 0.74 | 0.85 |
| rsv_inp_4 | 81 | 1.69 | 0.64 | 0.5 | 0.87 |
| dife_inp_1 | 92 | 0.3 | 0.24 | 0.61 | 0.65 |
| dife_inp_1_mutant | 87 | n/a | n/a | n/a | 0.71 |
| dife_inp_2 | 94 | 0.91 | 0.39 | 0.54 | 0.64 |
| dife_inp_2_mutant | 95 | n/a | n/a | n/a | 0.79 |
| dife_inp_3 | 90 | 0.54 | 0.31 | 0.72 | 0.76 |
| dife_inp_3_mutant | 92 | n/a | n/a | n/a | 0.89 |
| dife_inp_4 | 88 | 1.04 | 0.77 | 0.32 | 0.85 |
| dife_inp_5 | 90 | 0.82 | 0.67 | 0.39 | 0.71 |
| dife_inp_6 | 93 | 0.77 | 0.39 | 0.99 | 0.92 |
| dife_inp_7 | 95 | 0.4 | 0.27 | 0.64 | 0.68 |
| dife_inp_8 | 90 | 0.72 | 0.62 | 0.31 | 0.8 |
| Di-Fe_86 | 84 | 1.97 | 0.89 | 0.4 | 0.9 |
| Di-Fe_56 | 84 | 2.28 | 0.74 | 0.46 | 0.87 |
| EFhand_inp_1 | 87 | 0.86 | 0.82 | 0.29 | 0.69 |
| EFhand_inp_2 | 87.5 | 1.7 | 0.3 | 0.8 | 0.7 |
| EFhand_hal_1 | 82.2 | 1.42 | 0.59 | 0.36 | 0.52 |
| EFhand_hal_2 | 82.8 | 0.76 | 0.47 | 0.55 | 0.73 |
| hcA_1 | 73 | 1.44 | 0.73 (2.23) | 0.75 (1.39) | 1.04 (1.97) |
| hcA_2 | 71 | 1.62 | 0.46 (1.74) | 0.46 (1.36) | 0.62 (2.02) |
| ksi_1 (AF) | 84 | 1.04 | 0.30 (0.30) | 0.30 (1.22) | 0.30 (1.20) |
| ksi_2 (AF) | 72 | 1.06 | 0.16 (0.22) | 0.43 (1.63) | 0.53 (1.65) |
| pdl1_inp_1 | 84 | 0.79 | 0.51 | 1 | 1.1 |
| trkA_56 | 89 | 2.53 | 2.06 | 1.15 | 2.34 |
| mdm2_hal_1 | 88.6 | 1.70 | 1.75 | 0.73 | 1.29 |
| mdm2_hal_2 | 84.1 | 1.95 | 0.83 | 0.59 | 0.63 |
| mdm2_hal_3 | 81.7 | 1.14 | 1.00 | 0.77 | 0.68 |

TABLE 4
Interface metrics of protein-binder designs
AlphaFold ™ inter-PAE, binder pLDDT, AF-Rosetta ™ ddG, and
target-aligned binder RMSD (Materials and Methods) for protein-
binder designs presented in this paper. Note that designs based off
of motifs are listed here and in Table 3, but the free hallucinations
are only shown here. pdl1_inp_1 and trkA_56 were not designed
using 2-chain hallucination, so there were no RF complex design
models to use for target-aligned binder RMSD calculations.

| | | Binder | AF-Rosetta ™ | Target-aligned |
|---|---|---|---|---|
| Design | Inter-PAE | pLDDT | ddG | binder RMSD |
| pdl1_inp_1 | 5.695 | 88.5 | −49.9 | N/A |
| trkA_56 | 8.428 | 88.4 | −51.8 | N/A |
| mdm2_hal_1 | 5.904 | 87.6 | −47.2 | 2.93 |
| mdm2_hal_2 | 4.822 | 89.7 | −45.8 | 3.36 |
| mdm2_hal_3 | 6.208 | 87.1 | −45.9 | 3.48 |
| trkA_freehal_1 | 6.40 | 87.4 | −32.5 | 3.87 |
| trkA_freehal_2 | 4.63 | 92.1 | −35.8 | 1.24 |
| pdl1_freehal_1 | 5.58 | 84.8 | −38.23 | 3.43 |
| pdl1_freehal_2 | 9.72 | 82.3 | −26.36 | 1.58 |
| pdl1_freehal_3 | 8.87 | 81.0 | −37.15 | 1.59 |

TABLE 5
Similarity of designs to proteins in the PDB
Designed proteins were compared to protein in the PDB for structural
and sequence similarity with TM-align (73) and blastp (87), respectively
(Materials and Methods). BLAST “% identity” refers to the number of identities
over the best HSP, normalized to the length of the query sequence (design).

| | TM-align to PDB | | BLAST to NR | | |
|---|---|---|---|---|---|
| Design | Top hit | TM-score | Top hit | E-value | % Identity |
| dife_inp_1 | 5vju_A | 0.88968 | None | NA | NA |
| dife_inp_1_mutant | 5vju_A | 0.87791 | None | NA | NA |
| dife_inp_2 | 7jic_B | 0.83846 | WP_000675503.1 | 2.24E−02 | 23.009 |
| dife_inp_2_mutant | 7jic_B | 0.81537 | None | NA | NA |
| dife_inp_3 | 1yo7_A | 0.8462 | None | NA | NA |
| dife_inp_3_mutant | 4phq_B | 0.82892 | None | NA | NA |
| dife_inp_4 | 6egc_A | 0.83817 | None | NA | NA |
| dife_inp_5 | 6egc_A | 0.85404 | None | NA | NA |
| dife_inp_6 | 5vjs_A | 0.80393 | None | NA | NA |
| dife_inp_7 | 5vjs_A | 0.84261 | None | NA | NA |
| dife_inp_8 | 5vju_A | 0.84708 | 2LFD_A | 9.30E−03 | 21.97 |
| rsv_inp_1 | 5a2q_G | 0.61088 | 1G2C_A | 5.40E−01 | 40.323 |
| rsv_inp_2 | 6apd_B | 0.63827 | XP_021434148.1 | 6.38E+00 | 29.688 |
| rsv_inp_3 | 5clr_A | 0.60218 | WP_120068072.1 | 1.24E+00 | 28.571 |
| rsv_inp_4 | 5g4y_A | 0.67448 | WP_159887573.1 | 5.70E+00 | 35.484 |
| trkA_56 | 2d4c_A | 0.79627 | None | NA | NA |
| rsvfv_hal_1 | 6ntr_D | 0.69371 | 3KPE_A | 8.90E−01 | 25.714 |
| rsvfv_hal_2 | 4dmg_A | 0.71137 | RZV56203.1 | 3.12E+00 | 20 |
| rsvfv_hal_3 | 4auk_A | 0.69259 | WP_154333053.1 | 4.53E+00 | 30.556 |
| rsvf-v_854 | 5wb0_F | 0.58339 | 1G2C_A | 5.80E−02 | 27.397 |
| rsvf-v_870 | 5csl_B | 0.67343 | 1G2C_A | 2.63E+00 | 27.419 |
| rsvf-v_828 | 6cp8_A | 0.62341 | None | NA | NA |
| rsvf-v_903 | 2x32_B | 0.62796 | 3KPE_A | 1.56E−01 | 32.258 |
| rsvf-v_1050 | 5wti_Z | 0.58795 | AIZ95772.1 | 3.16E−02 | 31.884 |
| rsvf-ii_141 | 6ivm_A | 0.68385 | HHG91166.1 | 6.16E+00 | 17.391 |
| rsvf-ii_171 | 5j0l_E | 0.85867 | AWV19065.1 | 3.23E−01 | 33.75 |
| rsvf-ii_118 | 2yfa_A | 0.73974 | CCW60917.1 | 1.54E+00 | 27.473 |
| rsvf-ii_29 | 4jeh_B | 0.77833 | RKX18559.1 | 2.83E−01 | 17.117 |
| rsvf-ii_158 | 2j0o_A | 0.86231 | WP_068486906.1 | 3.04E−01 | 29.091 |
| ace2_76 | 7jh6_A | 0.81391 | QIN87098.1 | 5.91E+00 | 20.619 |
| ace2_1157 | 2j0o_A | 0.76147 | WP_100023565.1 | 1.17E+00 | 23.894 |
| ace2_1007 | 5tqy_A | 0.79896 | None | NA | NA |
| ace2_1846 | 5iig_A | 0.81951 | None | NA | NA |
| ace2_600 | 4q2g_B | 0.72104 | EPE07190.1 | 2.41E+00 | 26.733 |
| ace2_109 | 3zcj_B | 0.80246 | ROL44962.1 | 1.58E−01 | 21.667 |
| hcA_1 | 2hb0_A | 0.76753 | WP_107852251.1 | 3.10E−01 | 30 |
| hcA_2 | 6ohh_B | 0.79433 | WP_021068970.1 | 6.97E+00 | 22.857 |
| ksi_1 (AF) | 5k59_B | 0.73486 | WP_147602516.1 | 4.22E−01 | 20.619 |
| ksi_2 (AF) | 1z8k_A | 0.58109 | KAF3849996.1 | 2.21E+00 | 25 |
| Di-Fe_86 | 6h2f_H | 0.76045 | None | NA | NA |
| Di-Fe_56 | 6ezv_X | 0.75196 | TGO06933.1 | 1.45E+00 | 18.644 |
| pdl1_inp_1 | 5ldz_F | 0.61099 | WP_071803821.1 | 3.28E−02 | 25.455 |
| EFhand_inp_1 | 4by5_B | 0.75179 | XP_020433196.1 | 1.38E−17 | 51.685 |
| EFhand_inp_2 | 1juo_A | 0.64636 | None | 9.52E−05 | 35.443 |
| EFhand_hal_1 | 2f8p_A | 0.72449 | XP_019463585.1 | 5.04E−02 | 35.593 |
| EFhand_hal_2 | 6afs_B | 0.67237 | WP_092746209.1 | 6.09E−02 | 23 |
| mdm2_hal_1 | 5h78_A | 0.77199 | None | NA | NA |
| mdm2_hal_2 | 1fjg_T | 0.77524 | XP_012788760.1 | 2.38E+00 | 26.786 |
| mdm2_hal_3 | 6w2v_B | 0.85863 | XP_030199201.1 | 7.14E+00 | 26.667 |
| trkA_freehal_1 | 5wyl_A | 0.74523 | WP_165006269.1 | 3.64E+00 | 24.615 |
| trkA_freehal_2 | 2oku_A | 0.82488 | None | NA | NA |
| pdl1_freehal_1 | 3q5d_A | 0.76861 | WP_132874866.1 | 7.67E+00 | 32.787 |
| pdl1_freehal_2 | 4jhc_A | 0.78276 | MSR05998.1 | 4.94E+00 | 25 |
| pdl1_freehal_3 | 2ygt_A | 0.75466 | PVH99412.1 | 2.20E+00 | 31.667 |

### REFERENCES

1. O. Khersonsky, A. M. Wollacott, L. Jiang, J. Dechancie, J. Betker, J. L. Gallaher, E. A. Althoff, A. Zanghellini, O. Dym, S. Albeck, K. N. Houk, D. S. Tawfik, D. Baker, Kemp elimination catalysts by computational enzyme design. 453 (2008), doi:10.1038/nature06879.
2. L. Jiang, E. A. Althoff, F. R. Clemente, L. Doyle, D. Rothlisberger, A. Zanghellini, J. L. Gallaher, J. L. Betker, F. Tanaka, C. F. Barbas, D. Hilvert, K. N. Houk, B. L. Stoddard, D. Baker, De Novo Computational Design of Retro-Aldol Enzymes. Science. 319, 1387-1391 (2008).
3. J. B. Siegel, A. Zanghellini, H. M. Lovick, G. Kiss, A. R. Lambert, J. L. St. Clair, J. Gallaher, D. Hilvert, M. H. Gelb, B. L. Stoddard, K. N. Houk, F. E. Michael, D. Baker, Computational Design of an Enzyme Catalyst for a Stereoselective Bimolecular Diels-Alder Reaction. Science. 329 (2010), doi:10.1126/science.1190239.
4. L. Cao, B. Coventry, I. Goreshnik, B. Huang, J. S. Park, K. M. Jude, I. Markovi6, R. U. Kadam, K. H. G. Verschueren, K. Verstraete, S. T. R. Walsh, N. Bennett, A. Phal, A. Yang, L. Kozodoy, M. DeWitt, L. Picton, L. Miller, E.-M. Strauch, N. D. DeBouver, A. Pires, A. K. Bera, S. Halabiya, B. Hammerson, W. Yang, S. Bernard, L. Stewart, I. A. Wilson, H. Ruohola-Baker, J. Schlessinger, S. Lee, S. N. Savvides, K. C. Garcia, D. Baker, Design of protein binding proteins from target structure alone. Nature (2022), doi:10.1038/s41586-022-04654-9.
5. A. A. Chevalier, D. Silva, G. J. Rocklin, R. Derrick, R. Vergara, P. Murapa, S. M. Bernard, L. Zhang, G. Yao, C. D. Bahl, S. Miyashita, I. Goreshnik, T. James, M. Bryan, D. A. Fernindez-velasco, L. Stewart, M. Dong, X. Huang, Massively parallel de novo protein design for targeted therapeutics. Nat. Publ. Group (2017), doi:10.1038/nature23912.
6. E. Procko, G. Y. Berguig, B. W. Shen, Y. Song, S. Frayo, A. J. Convertine, D. Margineantu, G. Booth, B. E. Correia, Y. Cheng, W. R. Schief, D. M. Hockenbery, O. W. Press, B. L. Stoddard, P. S. Stayton, D. Baker, A Computationally Designed Inhibitor of an Epstein-Barr Viral Bcl-2 Protein Induces Apoptosis in Infected Cells. Cell. 157, 1644-1656 (2014).
7. B. E. Correia, J. T. Bates, R. J. Loomis, G. Baneyx, C. Carrico, J. G. Jardine, P. Rupert, C. Correnti, O. Kalyuzhniy, V. Vittal, M. J. Connell, E. Stevens, A. Schroeter, M. Chen, S. MacPherson, A. M. Serra, Y. Adachi, M. A. Holmes, Y. Li, R. E. Klevit, B. S. Graham, R. T. Wyatt, D. Baker, R. K. Strong, J. E. Crowe, P. R. Johnson, W. R. Schief, Proof of principle for epitope-focused vaccine design. Nature. 507, 201-206 (2014).
8. D.-A. Silva, S. Yu, U. Y. Ulge, J. B. Spangler, K. M. Jude, C. Lab§ o-Almeida, L. R. Ali, A. Quijano-Rubio, M. Ruterbusch, I. Leung, T. Biary, S. J. Crowley, E. Marcos, C. D. Walkey, B. D. Weitzner, F. Pardo-Avila, J. Castellanos, L. Carter, L. Stewart, S. R. Riddell, M. Pepper, G. J. L. Bernardes, M. Dougan, K. C. Garcia, D. Baker, De novo design of potent and selective mimics of IL-2 and IL-15. Nature. 565, 186-191 (2019).
9. F. Sesterhenn, C. Yang, J. Bonet, J. T. Cramer, X. Wen, Y. Wang, C.-I. Chiang, L. A. Abriata, I. Kucharska, G. Castoro, S. S. Vollers, M. Galloux, E. Dheilly, S. Rosset, P. Corthesy, S. Georgeon, M. Villard, C.-A. Richard, D. Descamps, T. Delgado, E. Oricchio, M.-A. Rameix-Welti, V. Mis, S. Ervin, J.-F. Eleou6t, S. Riffault, J. T. Bates, J.-P. Julien, Y. Li, T. Jardetzky, T. Krey, B. E. Correia, De novo protein design enables the precise induction of RSV-neutralizing antibodies. Science. 368 (2020), doi:10.1126/science.aay5051.
10. C. Yang, F. Sesterhenn, J. Bonet, E. A. van Aalen, L. Scheller, L. A. Abriata, J. T. Cramer, X. Wen, S. Rosset, S. Georgeon, T. Jardetzky, T. Krey, M. Fussenegger, M. Merkx, B. E. Correia, Bottom-up de novo design of functional proteins with complex structural features. Nat. Chem. Biol., 1-9 (2021).
11. J. Yang, I. Anishchenko, H. Park, Z. Peng, S. Ovchinnikov, D. Baker, Improved protein structure prediction using predicted interresidue orientations. Proc. Natl. Acad. Sci. (2020), doi:10.1073/pnas.1914677117.
12. I. Anishchenko, S. J. Pellock, T. M. Chidyausiku, T. A. Ramelot, S. Ovchinnikov, J. Hao, K. Bafna, C. Norn, A. Kang, A. K. Bera, F. DiMaio, L. Carter, C. M. Chow, G. T. Montelione, D. Baker, De novo protein design by deep network hallucination. Nature. 600, 547-552 (2021).
13. C. Norn, B. I. M. Wicky, D. Juergens, S. Liu, D. Kim, D. Tischer, B. Koepnick, I. Anishchenko, F. Players, D. Baker, S. Ovchinnikov, Protein sequence design by conformational landscape optimization. Proc. Natl. Acad. Sci. 118 (2021), doi:10.1073/pnas.2017228118.
14. D. Tischer, S. Lisanza, J. Wang, R. Dong, I. Anishchenko, L. F. Milles, S. Ovchinnikov, D. Baker, bioRxiv, in press, doi:10.1101/2020.11.29.402743.
15. M. Baek, F. DiMaio, I. Anishchenko, J. Dauparas, S. Ovchinnikov, G. R. Lee, J. Wang, Q. Cong, L. N. Kinch, R. D. Schaeffer, C. Millin, H. Park, C. Adams, C. R. Glassman, A. DeGiovanni, J. H. Pereira, A. V. Rodrigues, A. A. van Dijk, A. C. Ebrecht, D. J. Opperman, T. Sagmeister, C. Buhlheller, T. Pavkov-Keller, M. K. Rathinaswamy, U. Dalwadi, C. K. Yip, J. E. Burke, K. C. Garcia, N. V. Grishin, P. D. Adams, R. J. Read, D. Baker, Accurate prediction of protein structures and interactions using a three-track neural network. Science (2021), doi:10.1 126/science.abj8754.
16. J. Devlin, M.-W. Chang, K. Lee, K. Toutanova, BERT: Pre-training of Deep Bidirectional Transformers for Language Understanding. ArXiv181004805 Cs (2019) (available at arxiv.org/abs/1810.04805).
17. R. A. Yeh, C. Chen, T. Y. Lim, A. G. Schwing, M. Hasegawa-Johnson, M. N. Do, Semantic Image Inpainting with Deep Generative Models. ArXiv160707539 Cs (2017) (available at arxiv.org/abs/1607.07539).
18. J. Jumper, R. Evans, A. Pritzel, T. Green, M. Figurnov, O. Ronneberger, K. Tunyasuvunakool, R. Bates, A. Zidek, A. Potapenko, A. Bridgland, C. Meyer, S. A. A. Kohl, A. J. Ballard, A. Cowie, B. Romera-Paredes, S. Nikolov, R. Jain, J. Adler, T. Back, S. Petersen, D. Reiman, E. Clancy, M. Zielinski, M. Steinegger, M. Pacholska, T. Berghammer, S. Bodenstein, D. Silver, O. Vinyals, A. W. Senior, K. Kavukcuoglu, P. Kohli, D. Hassabis, Highly accurate protein structure prediction with AlphaFold. Nature. 596, 583-589 (2021).
19. R. Chowdhury, N. Bouatta, S. Biswas, C. Rochereau, G. M. Church, P. K. Sorger, M. AlQuraishi, Single-sequence protein structure prediction using language models from deep learning, 22.
20. K. T. Simons, R. Bonneau, I. Ruczinski, D. Baker, Ab initio protein structure prediction of CASP III targets using ROSETTA. Proteins Struct. Funct. Bioinforma. 37, 171-176 (1999).
21. T.-E. Kim, K. Tsuboyama, S. Houliston, C. M. Martell, C. M. Phoumyvong, H. K. Haddox, C. H. Arrowsmith, G. J. Rocklin, Dissecting the stability determinants of a challenging de novo protein fold using massively parallel design and experimentation (2021), p. 2021.12.17.472837, doi:10.1101/2021.12.17.472837.
22. M. A. Pak, K. A. Markhieva, M. S. Novikova, D. S. Petrov, I. S. Vorobyev, E. S. Maksimova, F. A. Kondrashov, D. N. Ivankov, Using AlphaFold to predict the impact of single mutations on protein stability and function (2021), p. 2021.09.19.460937, doi:10.1101/2021.09.19.460937.
23. J. J. Mousa, N. Kose, P. Matta, P. Gilchuk, J. E. Crowe, A novel pre-fusion conformation-specific neutralizing epitope on the respiratory syncytial virus fusion protein. Nat. Microbiol. 2, 1-8 (2017).
24. T. W. Linsky, R. Vergara, N. Codina, J. W. Nelson, M. J. Walker, W. Su, C. O. Barnes, T.-Y. Hsiang, K. Esser-Nobis, K. Yu, Z. B. Reneer, Y. J. Hou, T. Priya, M. Mitsumoto, A. Pong, U. Y. Lau, M. L. Mason, J. Chen, A. Chen, T. Berrocal, H. Peng, N. S. Clairmont, J. Castellanos, Y.-R. Lin, A. Josephson-Day, R. S. Baric, D. H. Fuller, C. D. Walkey, T. M. Ross, R. Swanson, P. J. Bjorkman, M. Gale, L. M. Blancas-Mejia, H.-L. Yen, D.-A. Silva, De novo design of potent and resilient hACE2 decoys to neutralize SARS-CoV-2. Science (2020), doi:10.1126/science.abe0075.
25. F. Frolow, A. J. Kalb (Gilboa), J. Yariv, Structure of a unique twofold symmetric haem-binding site. Nat. Struct. Biol. 1, 453-460 (1994).
26. A. Lombardi, F. Pirro, O. Maglio, M. Chino, W. F. DeGrado, De Novo Design of Four-Helix Bundle Metalloproteins: One Scaffold, Diverse Reactivities. Acc. Chem. Res. 52, 1148-1159 (2019).
27. J. R. Calhoun, F. Nastri, O. Maglio, V. Pavone, A. Lombardi, W. F. DeGrado, Artificial diiron proteins: From structure to function. Pept. Sci. 80, 264-278 (2005).
28. A. M. Keech, N. E. L. Brun, M. T. Wilson, S. C. Andrews, G. R. Moore, A. J. Thomson, Spectroscopic Studies of Cobalt(II) Binding to Escherichia coli Bacterioferritin*. J. Biol. Chem. 272, 422-429 (1997).
29. E. N. G. Marsh, W. F. DeGrado, Noncovalent self-assembly of a heterotetrameric diiron protein. Proc. Natd. Acad. Sci. 99, 5150-5154 (2002).
30. M. Yinez, J. Gil-Longo, M. Campos-Toimil, in Calcium Signaling, Md. S. Islam, Ed. (Springer Netherlands, Dordrecht, 2012; doi.org/10.1007/978-94-007-2888-2_19), Advances in Experimental Medicine and Biology, pp. 461-482.
31. S. J. Caldwell, I. C. Haydon, N. Piperidou, P.-S. Huang, M. J. Bick, H. S. Sjostrom, D. Hilvert, D. Baker, C. Zeymer, Tight and specific lanthanide binding in a de novo TIM barrel with a large internal cavity designed by symmetric domain fusion. Proc. Natl. Acad. Sci. 117, 30362-30369 (2020).
32. C. U. Kim, H. Song, B. S. Avvaru, S. M. Gruner, S. Park, R. McKenna, Tracking solvent and protein movement during CO2 release in carbonic anhydrase II crystals. Proc. Natl. Acad. Sci. 113, 5257-5262 (2016).
33. M. R. Badger, G. D. Price, The Role of Carbonic Anhydrase in Photosynthesis. Annu. Rev. Plant Physiol. 45, 369-92 (1994).
34. P. Mirjafari, K. Asghari, N. Mahinpey, Investigating the Application of Enzyme Carbonic Anhydrase for CO2 Sequestration Purposes. Ind. Eng. Chem. Res. 46, 921-926 (2007).
35. H.-S. Cho, N.-C. Ha, G. Choi, H.-J. Kim, D. Lee, K. S. Oh, K. S. Kim, W. Lee, K. Y. Choi, B.-H. Oh, Crystal Structure of A5-3-Ketosteroid Isomerase from Pseudomonas testosteroni in Complex with Equilenin Settles the Correct Hydrogen Bonding Scheme for Transition State Stabilization*. J. Biol. Chem. 274, 32863-32868 (1999).
36. R. Pascolutti, X. Sun, J. Kao, R. L. Maute, A. M. Ring, G. R. Bowman, A. C. Kruse, Structure and Dynamics of PD-L1 and an Ultra-High-Affinity PD-1 Receptor Mutant. Structure. 24, 1719-1728 (2016).
37. R. L. Maute, S. R. Gordon, A. T. Mayer, M. N. McCracken, A. Natarajan, N. G. Ring, R. Kimura, J. M. Tsai, A. Manglik, A. C. Kruse, S. S. Gambhir, I. L. Weissman, A. M. Ring, Engineering high-affinity PD-1 variants for optimized immunotherapy and immuno-PET imaging. Proc. Natl. Acad. Sci. 112, E6506-E6514 (2015).
38. C. Wiesmann, M. H. Ultsch, S. H. Bass, A. M. de Vos, Crystal structure of nerve growth factor in complex with the ligand-binding domain of the TrkA receptor. Nature. 401, 184-188 (1999). 39. I. R. Humphreys, J. Pei, M. Baek, A. Krishnakumar, I. Anishchenko, S. Ovchinnikov, J. Zhang, T. J. Ness, S. Banjade, S. R. Bagde, V. G. Stancheva, X.-H. Li, K. Liu, Z. Zheng, D. J. Barrero, U. Roy, J. Kuper, I. S. Fernindez, B. Szakal, D. Branzei, J. Rizo, C. Kisker, E. C. Greene, S. Biggins, S. Keeney, E. A. Miller, J. C. Fromme, T. L. Hendrickson, Q. Cong, D. Baker, Computed structures of core eukaryotic protein complexes. Science. 0, eabm4805.
40. K. Tunyasuvunakool, J. Adler, Z. Wu, T. Green, M. Zielinski, A. Zidek, A. Bridgland, A. Cowie, C. Meyer, A. Laydon, S. Velankar, G. J. Kleywegt, A. Bateman, R. Evans, A. Pritzel, M. Figurnov, O. Ronneberger, R. Bates, S. A. A. Kohl, A. Potapenko, A. J. Ballard, B. Romera-Paredes, S. Nikolov, R. Jain, E. Clancy, D. Reiman, S. Petersen, A. W. Senior, K. Kavukcuoglu, E. Birney, P. Kohli, J. Jumper, D. Hassabis, Highly accurate protein structure prediction for the human proteome. Nature (2021), doi:10.1038/s41586-021-03828-1.
41. H. M. Berman, J. Westbrook, Z. Feng, G. Gilliland, T. N. Bhat, H. Weissig, I. N. Shindyalov, P. E. Bourne, The Protein Data Bank. Nucleic Acids Res. 28, 235-242 (2000).
42. J. Ingraham, V. K. Garg, R. Barzilay, T. Jaakkola, Generative models for graph-based protein design, 10 (2019).
43. A. Strokach, D. Becerra, C. Corbi-Verge, A. Perez-Riba, P. M. Kim, Fast and Flexible Protein Design Using Deep Graph Neural Networks. Cell Syst. 11, 402-411.e4 (2020).
44. S. Biswas, G. Khimulya, E. C. Alley, K. M. Esvelt, G. M. Church, Low-N protein engineering with data-efficient deep learning. Nat. Methods. 18, 389-396 (2021).
45. D. Repecka, V. Jauniskis, L. Karpus, E. Rembeza, J. Zrimec, S. Poviloniene, I. Rokaitis, A. Laurynenas, W. Abuajwa, O. Savolainen, R. Meskys, M. K. M. Engqvist, A. Zelezniak, Expanding functional protein sequence space using generative adversarial networks. bioRxiv, 789719 (2019).
46. J.-E. Shin, A. J. Riesselman, A. W. Kollasch, C. McMahon, E. Simon, C. Sander, A. Manglik, A. C. Kruse, D. S. Marks, Protein design and variant prediction using autoregressive generative models. Nat. Commun. 12, 1-11 (2021).
47. Z. Wu, K. E. Johnston, F. H. Arnold, K. K. Yang, Protein sequence design with deep generative models. Curr. Opin. Chem. Biol. 65, 18-27 (2021).
48. N. Anand-Achim, R. R. Eguchi, A. Derry, R. B. Altman, P.-S. Huang, “Protein sequence design with a learned potential” (preprint, Bioinformatics, 2020), doi:10.1101/2020.01.06.895466.
49. A. Madani, B. Krause, E. R. Greene, S. Subramanian, B. P. Mohr, J. M. Holton, J. L. Olmos, C. Xiong, Z. Z. Sun, R. Socher, J. S. Fraser, N. Naik, bioRxiv, in press, doi:10.1101/2021.07.18.452833.
50. S. Ovchinnikov, P.-S. Huang, Structure-based protein design with deep learning. Curr. Opin. Chem. Biol. 65, 136-144 (2021).
51. N. Anand, R. Eguchi, P.-S. Huang, Fully differentiable full-atom protein backbone generation (2019) (available at openreview.net/forum?id=SJxnVL8YOV).
52. R. R. Eguchi, N. Anand, C. A. Choe, P.-S. Huang, bioRxiv, in press, doi:10.1101/2020.08.07.242347.
53. Z. Lin, T. Sercu, Y. LeCun, A. Rives, Deep generative models create new and diverse protein structures, 17.
54. M. Jendrusch, J. O. Korbel, S. K. Sadiq, bioRxiv, in press, doi:10.1101/2021.10.11.463937.
55. L. Moffat, J. G. Greener, D. T. Jones, bioRxiv, in press, doi:10.1101/2021.08.24.457549.
56. L. Moffat, S. M. Kandathil, D. T. Jones, Design in the DARK: Learning Deep Generative Models for De Novo Protein Design (2022), p. 2022.01.27.478087, doi:10.1101/2022.01.27.478087.
57. Z. Li, S. P. Nguyen, D. Xu, Y. Shang, in 2017 IEEE 29th International Conference on Tools with Artificial Intelligence (ICTAI) (2017), pp. 1085-1091.
58. N. Anand, P. Huang, in Advances in Neural Information Processing Systems 31, S. Bengio, H. Wallach, H. Larochelle, K. Grauman, N. Cesa-Bianchi, R. Garnett, Eds. (Curran Associates, Inc., 2018; papers.nips.cc/paper/7978-generative-modeling-for-protein-structures.pdf), pp. 7494-7505.
59. L. Li, Y. Liu, J. Tao, M. Zhang, H. Pan, X. Xu, R. Tang, Surface Modification of Hydroxyapatite Nanocrystallite by a Small Amount of Terbium Provides a Biocompatible Fluorescent Probe. J. Phys. Chem. C. 112, 12219-12224 (2008).
60. I. Anishchenko, T. M. Chidyausiku, S. Ovchinnikov, S. J. Pellock, D. Baker, bioRxiv, in press, doi:10.1101/2020.07.22.211482.
61. E. Jang, S. Gu, B. Poole, Categorical Reparameterization with Gumbel-Softmax. ArXiv161101144 Cs Stat (2017) (available at arxiv.org/abs/1611.01144).
62. N. Bogard, J. Linder, A. B. Rosenberg, G. Seelig, A Deep Neural Network for Predicting and Engineering Alternative Polyadenylation. Cell. 178, 91-106.e23 (2019).
63. J. Linder, G. Seelig, Fast differentiable DNA and protein sequence optimization for molecular design. ArXiv200511275 Cs Stat (2020) (available at arxiv.org/abs/2005.11275).
64. D. P. Kingma, J. Ba, Adam: A Method for Stochastic Optimization. ArXiv14126980 Cs (2017) (available at arxiv.org/abs/1412.6980).
65. R. M. Rao, J. Liu, R. Verkuil, J. Meier, J. Canny, P. Abbeel, T. Sercu, A. Rives, bioRxiv, in press, doi:10.1101/2021.02.12.430858.
66. C. Hsu, R. Verkuil, J. Liu, Z. Lin, B. Hie, T. Sercu, A. Lerer, A. Rives, Learning inverse folding from millions of predicted structures (2022), p. 2022.04.10.487779, doi:10.1101/2022.04.10.487779.
67. N. Chennamsetty, V. Voynov, V. Kayser, B. Helk, B. L. Trout, Design of therapeutic proteins with enhanced stability. Proc. Natd. Acad. Sci. 106, 11937-11942 (2009).
68. S. K. Jha, A. Ramanathan, R. Ewetz, A. Velasquez, S. Jha, Protein Folding Neural Networks Are Not Robust. ArXiv210904460 Cs Q-Bio (2021) (available at arxiv.org/abs/2109.04460).
69. A. Ilyas, S. Santurkar, D. Tsipras, L. Engstrom, B. Tran, A. Madry, Adversarial Examples Are Not Bugs, They Are Features. ArXiv190502175 Cs Stat (2019) (available at arxiv.org/abs/1905.02175).
70. A. Demontis, M. Melis, M. Pintor, M. Jagielski, B. Biggio, A. Oprea, C. Nita-Rotaru, F. Roli, Why Do Adversarial Attacks Transfer? Explaining Transferability of Evasion and Poisoning Attacks. ArXiv180902861 Cs Stat (2019) (available at arxiv.org/abs/1809.02861).
71. B. Dang, M. Mravic, H. Hu, N. Schmidt, B. Mensa, W. F. DeGrado, SNAC-tag for sequence-specific chemical protein cleavage. Nat. Methods. 16, 319-322 (2019).
72. F. W. Studier, Protein production by auto-induction in high density shaking cultures. Protein Expr. Purif 41, 207-234 (2005).
73. Y. Zhang, J. Skolnick, TM-align: a protein structure alignment algorithm based on the TM-score. Nucleic Acids Res. 33, 2302-2309 (2005).
74. A. Jaegle, F. Gimeno, A. Brock, A. Zisserman, O. Vinyals, J. Carreira, Perceiver: General Perception with Iterative Attention. ArXiv210303206 Cs Eess (2021) (available at arxiv.org/abs/2103.03206).
75. W. Kabsch, A solution for the best rotation to relate two sets of vectors. Acta Crystallogr. A. 32, 922-923 (1976).
76. A. W. Senior, R. Evans, J. Jumper, J. Kirkpatrick, L. Sifre, T. Green, C. Qin, A. Zidek, A. W. R. Nelson, A. Bridgland, H. Penedones, S. Petersen, K. Simonyan, S. Crossan, P. Kohli, D. T. Jones, D. Silver, K. Kavukcuoglu, D. Hassabis, Improved protein structure prediction using potentials from deep learning. Nature, 1-5 (2020).
77. R. F. Alford, A. Leaver-Fay, J. R. Jeliazkov, M. J. O'Meara, F. P. DiMaio, H. Park, M. V. Shapovalov, P. D. Renfrew, V. K. Mulligan, K. Kappel, J. W. Labonte, M. S. Pacella, R. Bonneau, P. Bradley, R. L. Dunbrack, R. Das, D. Baker, B. Kuhlman, T. Kortemme, J. J. Gray, The Rosetta All-Atom Energy Function for Macromolecular Modeling and Design. J. Chem. Theory Comput. 13, 3031-3048 (2017).
78. D.-A. Silva, B. E. Correia, E. Procko, in ComputationalDesign ofLigand Binding Proteins, B. L. Stoddard, Ed. (Springer, New York, NY, 2016; doi.org/10.1007/978-1-4939-3569-7_17), Methods in Molecular Biology, pp. 285-304.
79. M. Steinegger, J. Soding, MMseqs2 enables sensitive protein sequence searching for the analysis of massive data sets. Nat. Biotechnol. 35, 1026-1028 (2017).
80. H. Park, P. Bradley, P. Greisen, Y. Liu, V. K. Mulligan, D. E. Kim, D. Baker, F. DiMaio, Simultaneous Optimization of Biomolecular Energy Functions on Features from Small Molecules and Macromolecules. J. Chem. Theory Comput. 12, 6201-6212 (2016).
81. V. Hornak, R. Abel, A. Okur, B. Strockbine, A. Roitberg, C. Simmerling, Comparison of multiple Amber force fields and development of improved protein backbone parameters. Proteins Struct. Funct. Bioinforma. 65, 712-725 (2006).
82. R. Pascolutti, X. Sun, J. Kao, R. L. Maute, A. M. Ring, G. R. Bowman, A. C. Kruse, Structure and Dynamics of PD-L1 and an Ultra-High-Affinity PD-1 Receptor Mutant. Structure. 24, 1719-1728 (2016).
83. J. S. McLellan, M. Chen, A. Kim, Y. Yang, B. S. Graham, P. D. Kwong, Structural basis of respiratory syncytial virus neutralization by motavizumab. Nat. Struct. Mol. Biol. 17, 248-250 (2010).
84. J. Shang, G. Ye, K. Shi, Y. Wan, C. Luo, H. Aihara, Q. Geng, A. Auerbach, F. Li, Structural basis of receptor recognition by SARS-CoV-2. Nature. 581, 221-224 (2020).
85. J. L. Fallon, F. A. Quiocho, A Closed Compact Structure of Native Ca2+-Calmodulin. Structure. 11, 1303-1307 (2003).
86. P. H. Kussie, S. Gorina, V. Marechal, B. Elenbaas, J. Moreau, A. J. Levine, N. P. Pavletich, Structure of the MDM2 Oncoprotein Bound to the p53 Tumor Suppressor Transactivation Domain. Sci. New Ser. 274, 948-953 (1996).
87. S. F. Altschul, W. Gish, W. Miller, E. W. Myers, D. J. Lipman, Basic local alignment search tool. J. Mol. Biol. 215, 403-410 (1990).

We claim:

1. A polypeptide comprising an amino acid sequence at least 75%, 80%, 85%, 90%, 91%, 92%, 93% 94%, 95%, 96%, 97% 98%, 99% or 100% identical to the amino acid sequence selected from SEQ TD NO:1-15, wherein any N-terminal methionine residues are optional and may be present or absent.

2. The polypeptide of claim 1, comprising an amino acid sequence at least 75%, 80%, 85%, 90%, 91%, 92%, 93%, 94%, 95%, 96%, 97%, 98%, 99% or 100% identical to the amino acid sequence selected from SEQ ID NO: 1-8, wherein the polypeptide binds to Co²⁺.

3. The polypeptide of claim 1, comprising an amino acid sequence at least 75% 80%, 85%, 90%, 91%, 92%, 93%, 94%, 95%, 96%, 97%, 98%, 99% or 100% identical to the amino acid sequence selected from SEQ ID NO: 1-3, wherein the polypeptide binds to Co²⁺.

4. The polypeptide of claim 2, wherein bold-faced residues are conserved in the polypeptide.

| SEQ ID | |
|---|---|
| NO. | Name/Sequence |
| 1 | >dife_inp__1 |
| | EEEEARRLLERAAELELVAINQYRRAAEEAAERAAEGDEEARELAEVERRESIDEMKHAERLR |
| | ELLARGLSPEDRPELRRALEEILRDEEGHARRYAELAEEFGSPEARELAALELDGAKLYREAL |
| | ELLS |
| 2 | >dife_inp_2 |
| | EEAKELLKELVALELDGAKRYREALEEAKAMGASEEERAVLEEILRDEEGHARRLRKYLEAGD |
| | LEGAAELARKESIDEMKHAERFAELGLPEELRELELVAINQYRKAAEALR |
| 3 | >dife_inp_3 |
| | DEELRRELERILRDEEGHAKRAREALEEAERLGLSDEVRRAAEELAALELDGAKLAREAIEEG |
| | LSEEALERYAELELVAINQYRRLAELLEREGAPEELAERFRRESIEEMKHAERLRELL |
| 4 | >dife_inp_4 |
| | EEEELEELAKELEKILRDEEGHLRKLKEALAEGLGDAEEAAELFRAESIDEMKHAEELAKLLK |
| | KGGLDPELRELLEELAELELVAINQYREAAEAAAEAAENGSEEARAAAREALEEALALELDGA |
| | KLARAALEAVEKLL |
| 5 | >dife_inp_5 |
| | LEEARAELAAILRDEEGHIRRAAAGGLTAEEFRKESIDEMKHAEKVLELAEEAAAKGDEEAAE |
| | AAAEAAELELVAINQYRAAAAAGPSEEARAALLALELDGAKLYREALAALEA |
| 6 | >dife_inp_6 |
| | DEEVEEARRALERILRDEEGHARRYEEMAEEARRMGGDEELARALEELAALELDGAKLAREAL |
| | EAVEAGDLEKAREAAEEYAELELVAINQYRELAKEYPDFAEEARRESIDEMKHAEELRELVEE |
| | L |
| 7 | >dife_inp_7 |
| | ERFEEAAELFRKESIDEMKHREELRELAEEDPEYAEIAEELAELELVAINQYGEAAEAAEEAA |
| | ENGSEEAREKALRALEEALALELDGAKRYREALEELERKGAPEEVRAALERILRDEEGHLRRL |
| | REALEELR |
| 8 | >dife_inp_8 |
| | TEELAAALEELLALELDGAKLYREALAAAPSPEERAELERILRDEEGHLAKLRELLEKFKAGA |
| | SKEELKEAAELFRRESIDEMKHAERLRELAEDPDLSPEDRAAAEEAAELELVAINQYGEAAEE |
| | AARAAE |

5. The polypeptide of claim 1, comprising an amino acid sequence at least 75%, 80%, 85%, 90%, 91%, 92%, 93%, 94%, 95%, 96%, 97%, 98%, 99%, or 100% identical to the amino acid sequence selected from SEQ ID NO:9-10, wherein the polypeptide binds to Ca²⁺.

6. The polypeptide of claim 1, comprising an amino acid sequence at least 75%, 80%, 85%, 90%, 91%, 92%, 93%, 94%, 95%, 96%, 97%, 98%, 99%, or 100% identical to the amino acid sequence selected from SEQ ID NO:11-14, wherein the polypeptide binds to Rous sarcoma virus (RSV) F protein.

7. The polypeptide of claim 1, comprising an amino acid sequence at least 75%, 80%, 85%, 90%, 91%, 92%, 93%, 94%, 95%, 96%, 97%, 98%, 99%, or 100% identical to the amino acid sequence of SEQ ID NO:15, wherein the polypeptide binds to Tropomyosin receptor kinase A (TrkA).

8. The polypeptide of claim 1, comprising an amino acid sequence at least 75%, 80%, 85%, 90%, 91%, 92%, 93%, 94%, 95%, 96%, 97%, 98%, 99%, or 100% identical to the amino acid sequence of SEQ ID NO:22, wherein the polypeptide binds to programmed cell death ligand 1 (PD-L1).

9. The polypeptide of claim 1, wherein substitutions relative to the reference sequence are conservative amino acid substitutions.

10. A fusion protein, comprising the polypeptide of claim 1 fused to a further functional domain.

11. A nucleic acid encoding the polypeptide of claim 1.

12. An expression vector comprising the nucleic acid of claim 11 operatively linked to a promoter.

13. A host cell comprising the nucleic acid of claim 11 or the expression vector of claim 12.

14. A method for treating cancer, comprising administering to a subject in need thereof an amount effective to treat the cancer of the polypeptide of claim 7.

15. A method for treating cancer, comprising administering to a subject in need thereof an amount effective to treat the cancer of the polypeptide of claim 8.

16. A method for generating an immune response in a subject, comprising administering to a subject in need thereof an amount effective to generate an immune response in the subject of the polypeptide of claim 6.

17. A calcium imaging probe, comprising the polypeptide of claim 5.

18. An electron microscopy stain, comprising the polypeptide of claim 2.


SEQUENCE LISTING (22 sequences)

SEQ ID NO: 1 - protein, 130 aa, synthetic construct
EEEEARRLLERAAELELVAINQYRRAAEEAAERAAEGDEEARELAEVFRRESIDEMKHAERLRELLARGLSPEDRPELRRALEEILRDEEGHARRYAELAEEFGSPEARELAALELDGAKLYREALELLS

SEQ ID NO: 2 - protein, 113 aa, synthetic construct
EEAKELLKELVALELDGAKRYREALEEAKAMGASEEERAVLEEILRDEEGHARRLRKYLEAGDLEGAAELARKESIDEMKHAERFAELGLPEELRELELVAINQYRKAAEALR

SEQ ID NO: 3 - protein, 121 aa, synthetic construct
DEELRRELERILRDEEGHAKRAREALEEAERLGLSDEVRRAAEELAALELDGAKLAREAIEEGLSEEALERYAELELVAINQYRRLAELLEREGAPEELAERFRRESIEEMKHAERLRELL

SEQ ID NO: 4 - protein, 140 aa, synthetic construct
EEEELEELAKELEKILRDEEGHLRKLKEALAEGLGDAEEAAELFRAESIDEMKHAEELAKLLKKGGLDPELRELLEELAELELVAINQYREAAEAAAEAAENGSEEARAAAREALEEALALELDGAKLARAALEAVEKLL

SEQ ID NO: 5 - protein, 115 aa, synthetic construct
LEEARAELAAILRDEEGHIRRAAAGGLTAEEFRKESIDEMKHAEKVLELAEEAAAKGDEEAAEAAAEAAELELVAINQYRAAAAAGPSEEARAALLALELDGAKLYREALAALEA

SEQ ID NO: 6 - protein, 127 aa, synthetic construct
DEEVEEARRALERILRDEEGHARRYEEMAEEARRMGGDEELARALEELAALELDGAKLAREALEAVEAGDLEKAREAAEEYAELELVAINQYRELAKEYPDFAEEARRESIDEMKHAEELRELVEEL

SEQ ID NO: 7 - protein, 134 aa, synthetic construct
ERFEEAAELFRKESIDEMKHREELRELAEEDPEYAEIAEELAELELVAINQYGEAAEAAEEAAENGSEEAREKALRALEEALALELDGAKRYREALEELERKGAPEEVRAALERILRDEEGHLRRLREALEELR

SEQ ID NO: 8 - protein, 132 aa, synthetic construct
TEELAAALEELLALELDGAKLYREALAAAPSPEERAELERILRDEEGHLAKLRELLEKFKAGASKEELKEAAELFRRESIDEMKHAERLRELAEDPDLSPEDRAAAEEAAELELVAINQYGEAAEEAARAAE

SEQ ID NO: 9 - protein, 89 aa, synthetic construct
DEEVEKLFSLFDKDGDGTITTKELGTVFKEVAKKTGKDLPFADEEEAAKLINEVDADGNGTIDFPEFLTMYKYLEENGVLDELLEALEG

SEQ ID NO: 10 - protein, 79 aa, synthetic construct
MEWMTREEFKEWLEKALEDPELPISPEEAELLLELPEEELQELFEEADKDGDGTITREELEEYQKKLKELAEELLEEKK

SEQ ID NO: 11 - protein, 62 aa, synthetic construct
SKKLKLYLNGKKGGSESDVNKAKSALLSTNKAVVSFSGDPELKGKEYVVELKNGKLYLKPLE

SEQ ID NO: 12 - protein, 64 aa, synthetic construct
LSKEELKKALKSPEARNKAKSALLSTNKAVVSLDPDDGKTYLVSKKNGKLKVFKNGKLLVSLPL

SEQ ID NO: 13 - protein, 63 aa, synthetic construct
SGGGKVNGKPISEEDRNKAKSALLSTNKAVVSVNGKEYTATKDPSTGKIVFFKNGKLVGSFPL

SEQ ID NO: 14 - protein, 62 aa, synthetic construct
KKLKLTDDGLKLSEEERNKAKSALLSTNKAVVSFKSKKDGKNGLLVVSKKNGKLKVLSKLKK

SEQ ID NO: 15 - protein, 111 aa, synthetic construct
DLDIIRRAAAELKRYEEKAEEFKEHLFKQVVRLIVTPEYQANPEKLEEVIQKIKDLEEEVEKKKEELFKEVVRAIVTGDHALAERVIERARELIEEIEKKKKEISEEVELG

SEQ ID NO: 16 - protein, 11 aa, synthetic construct
LEAFEKALKEM

SEQ ID NO: 17 - protein, 4 aa, synthetic construct
LEAF

SEQ ID NO: 18 - protein, 5 aa, synthetic construct
LEAFS

SEQ ID NO: 19 - protein, 5 aa, synthetic construct
KFEMA

SEQ ID NO: 20 - protein, 9 aa, synthetic construct
LFEMAGCEL

SEQ ID NO: 21 - protein, 17 aa, synthetic construct
LEAFSALKFEMAGCELS

SEQ ID NO: 22 - protein, 110 aa, synthetic construct
DLEEFFEECLKEAEEVRKEVLEGKARIQIKESVRKEDPEGGTYVCGVISLGPKGGKFHVVFHKEDPEGKEETLARFEFKVEGGKVELLELKSKDPELKKLAEKYAEKLLE